(19) United States
(12) Patent Application Publication
(10) Pub. No.: US 2014/0088144 A1
(43) Pub. Date: Mar. 27, 2014

(54) INHIBITING MICROBIAL INFECTIONS

(71) Applicant: Susan M. Egan, Lawrence, KS (US)

(72) Inventor: Susan M. Egan, Lawrence, KS (US)

(73) Assignee: THE UNIVERSITY OF KANSAS, Lawrence, KS (US)

(21) Appl. No.: 14/035,461

(22) Filed: Sep. 24, 2013

Related U.S. Application Data
(60) Provisional application No. 61/744,384, filed on Sep. 25, 2012.

(51) Int. Cl. A61K 31/4709

(52) U.S. Cl. CPC A61K 31/4709
USPC 514/314; 435/252.1

(57) ABSTRACT

A method of inhibiting a microbial infection can include: providing a compound of the invention or prodrugs or pharmaceutically acceptable salts thereof; and administering the compound to a subject in a therapeutically effective amount to inhibit the microbial infection. The therapeutically effective amount can be sufficient to inhibit a biological activity of a transcriptional activator of the microbe. The inhibited transcriptional activator is an AraC bacterial transcriptional activator. The AraC bacterial transcriptional activator can be RhaS, RhaR, Rns, or VirF. The microbe can be selected from Vibrio, Pseudomonas, Enterotoxigenic E. coli, and Shigella.

### CROSS-REFERENCE

This patent application claims the benefit of U.S. Provisional Patent Application 61/744,384 filed Sep. 25, 2012, which is incorporated herein by specific reference in its entirety.

### GOVERNMENT SUPPORT

This invention was made with government support under contract No. R01GM55099 awarded by the National Institutes of Health. The government has certain rights in the invention.

### BACKGROUND

Microbial infections continue to be problematic even after the advent of numerous antimicrobials. Also, many microbes are capable of developing resistance to these antimicrobials. As such, it would be beneficial to develop new ways to control microbial infections, and to control such microbial infections with agents that are less susceptible to generate agent-resistance.

### SUMMARY

In one embodiment, a method of inhibiting a microbial infection can include: providing a compound of the invention or prodrugs or pharmaceutically acceptable salts thereof; and administering the compound to a subject in a therapeutically effective amount to inhibit the microbial infection. In one aspect, the therapeutically effective amount is sufficient to inhibit a biological activity of a transcriptional activator of the microbe. In one aspect, the inhibited transcriptional activator is an AraC bacterial transcriptional activator. In one aspect, the AraC bacterial transcriptional activator is RhaS, RhaR, Rns, or VirF. In one aspect, the microbe is selected from Vibrio, Pseudomonas, Enterotoxigenic E. coli, and Shigella. In one aspect, the method can include inhibiting diarrhea associated with the microbial infection. In one aspect, the method can include administration prior to infection with the microbe. In one aspect, the method can include administration after infection with the microbe. In one aspect, the compound has specific inhibition of AraC bacterial transcriptional activators over other transcriptional activators. In one aspect, the therapeutically effective amount is sufficient to inhibit a virulence factor of the microbe. In one aspect, the therapeutically effective amount is sufficient to inhibit microbe entry into a cell. In one aspect, the therapeutically effective amount is not toxic to the microbe. In one aspect, the compound sterically inhibits the transcriptional factor from binding with DNA. In one aspect, the therapeutically effective amount is sufficient to reduce expression of VirF-dependent virulence genes. In one aspect, the VirF-dependent virulence genes are selected from icsA, virB, icsB and ipaB. Here, inhibiting can be reducing compared to an instance where the compound is not administered, and inhibiting ranges from slight reduction to significant reduction; however, inhibition is not prevention. The inhibiting can be a treatment for a disease state or a prophylactic to inhibit the disease state without assertion that the disease state is completely prevented. Thus, inhibition does not include complete prevention, but can include a slight, medium, through significant reduction in comparison to without the compound. However, the compounds may entirely prevent the microbial infection.

In one embodiment, a method of inhibiting a transcriptional factor from binding DNA can include: providing a compound represented by Formula 1 or prodrugs or pharmaceutically acceptable salts thereof; and administering the compound to a transcriptional factor in a therapeutically effective amount to inhibit the transcriptional factor from binding DNA.

In one embodiment, a method of inhibiting a microbe from entering a cell of a subject can include: providing a compound represented by Formula 1 or prodrugs or pharmaceutically acceptable salts thereof; and administering the compound to the subject having the cell in a therapeutically effective amount to inhibit the microbe from entering the cell.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 shows reporter fusions used in high-throughput screen. Top Panel: Primary screen, RhaS-activated rhaB-lacZ fusion, shown in uninduced, (−)rhamnose state (left) and induced (+)rhamnose state (right). Bottom Panel: Secondary screen, LacI-repressed hts-lacZ control fusion, shown in uninduced, (−)IPTG state (left) and induced (+)IPTG state (right). Gray rectangles: RhaS and Lad (lacO) binding sites. Gray arrows: lacZ gene expressed from rhaBAD or synthetic hts promoter. Thick black/gray line: Promoter region DNA. Right angle black arrow: transcription start sites. Rounded gray shapes: RNAP, RhaS or Lad proteins. Wavy lines: active transcription in the presence of inducers L-rhamnose (Rha) and IPTG. Compound 2, shown below, was identified as a hit from the high throughput screen, 1-ethyl-4-(nitromethyl)-3-quinolin-2-yl-4H-quinoline. Compound 1, shown below, is 1-butyl-4-(nitromethyl)-3-quinolin-2-yl-4H-quinoline.

FIG. 2 shows in vivo effects of Compound 1 on β-galactosidase expression from the reporter fusions hts-lacZ (SME3359, circles) and rhaB-lacZ activated by full-length RhaS (SME3634, squares) or RhaS(163-278) (RhaS-DBD, SME3635, triangles). Activity in the absence of inhibitor was set to 100% for each reporter fusion. Results are the average of three independent experiments with two replicates each.

FIG. 3 shows growth curves in the presence (+) and absence (−) of Compound 1. Strains SME3634 (rhaB-lacZ, pHG165rhaS) and SME3359 (hts-lacZ, pHG165lacI) were grown in the absence or presence of 44 mM OSSL_{—}051168. Results were averaged from two independent experiments with three replicates each.

FIGS. 4A-4B show the effect of Compound 1 on DNA binding by RhaS-GB1^{201b}. The DNA bound (assayed by EMSA) in the absence of inhibitor (Compound 1) was set to 100%, and binding in the presence of inhibitor is represented relative to that value. Inhibitor concentrations ranged from 650 to 10 mM, with serial two-fold dilutions. Results are the average of three independent experiments. Final concentrations of DNA and protein were 2.5 nM and 2.4 mM, respectively.

FIGS. 5A-5B shows the effect of Compound 1 on DNA binding by GB1^{b}-RhaR. The DNA bound (assayed by EMSA) in the absence of inhibitor (Compound 1) was set to 100%, and binding in the presence of inhibitor is represented relative to that value. Inhibitor concentrations ranged from 650 to 10 mM, with serial two-fold dilutions. Results are the average of three independent experiments. Final concentrations of DNA and protein were 1.3 nM and 2.2 mM, respectively.

FIGS. 6A-6B show the effect of Compound 1 on DNA binding by non-AraC family activators. DNA binding was assayed by EMSA. FIG. 6A shows CRP; at 480, 96 and 19 nM. DNA bound at 480 nM CRP in the absence of inhibitor was set to 100%, and all other values are represented relative to that value. Compound 1 was added at 1.3 mM where indicated. Final concentration of DNA was 0.84 nM. Results are the average of two independent experiments. FIG. 6B shows LacI; the DNA bound in the absence of inhibitor (Compound 1) was set to 100%, and binding in the presence of inhibitor is represented relative to that value Inhibitor concentrations ranged from 650 to 10 mM, with serial two-fold dilutions. Results are the average of three independent experiments. Final concentrations of DNA and protein were 90 nM and 5.6 mM, respectively.

FIG. 7 shows a model for Compound 1 inhibition. Our results indicate that Compound 1 inhibits by binding to the RhaS (and RhaR) proteins, and blocking its ability to bind to DNA. As a result, RhaS would not activate transcription in the presence of rhamnose. Labeling same as in FIG. 1 except small black rectangles are Compound 1.

FIG. 8 shows the effects of Compound 1 on in vivo activation by LacI, Rns and VirF. Expression of promoter-lacZ fusions regulated by the indicated activators, or the control Lad regulated fusion at the indicated concentrations of Compound 1. Average of three independent experiments, with two replicates in each experiment.

FIGS. 9A-9B show inhibition of MBP-Rns and MBP-VirF by Compound 1. Quantification of EMSA of MBP-Rns (FIG. 9A) or example EMSA of MBP-VirF (FIG. 9B) DNA binding with the indicated inhibitor concentrations [micromolar]. F is Free DNA.

FIGS. 10A-10B show the effect of Compound 1 on Growth of Strains for in vivo assays. Growth curves in E. coli in the presence (+) and absence (−) of Compound 1. Strains SME4021 (virB-lacZ) plus pHG165rns (left) or pHG165virF (right) and SME3358 (hts-lacZ) plus pHG165lacI (FIG. 10A & FIG. 10B) were grown in the absence or presence of 44 mM Compound 1.

FIG. 11 shows theoretical docking of Compound 1 in the structure of MarA [BL01], which serves as a model for the DNA binding domain of AraC family activators.

FIGS. 12A-12B show the effect of Compound 1 on Invasion of Epithelial Cells and Growth by Shigella.

FIG. 13 shows the effect of Compound 1 on Epithelial Cell Metabolic activity.

FIG. 14 shows inhibition of in vivo VirF activation in E. coli with Compound 1, where β-galactosidase (LacZ) activity was assayed at the indicated concentrations of inhibitor Compound 1 from two reporter fusions in E. coli: VirF-activated virB-lacZ (SME4382, circles) and LacI-repressed hts-lacZ (SME3359, triangles). VirF and Lad were each expressed from plasmid pHG165. Activity in the absence of Compound 1 was set to 100% in each case, and corresponded to approximately 950 Miller Units for virB-lacZ and 350 Miller Units for hts-lacZ. Results are the average of three independent experiments with two replicates each.

FIGS. 15A-15B show Compound 1 inhibition of in vitro DNA binding by VirF. FIG. 15A shows a representative EMSA gel picture. Black triangle represents decreasing concentrations of Compound 1, from 1.3 mM to 10 μM, with serial two-fold dilutions. FIG. 15B shows binding of purified VirF to a DNA fragment containing the VirF binding site from the virB promoter was assayed using electrophoretic mobility shift assays (EMSAs) with inhibitor Compound 1 concentrations from 10 to 650 μM (serial 2-fold dilutions). DNA and protein were at final concentrations of 2 and 300 nM, respectively. The DNA shifted (bound by VirF) was quantified, and the value at the lowest concentration of Compound 1 was set to 100%. Results are the average of three independent replicates.

FIGS. 16A-16B show a thermal shift assay showing that Compound 1 binds to VirF protein. The thermal denaturation of MBP-VirF (FIG. 16A) and MBP-NS1-NTD (FIG. 16B) were measured using the dye Sypro Orange. Assays were carried out in the absence (DMSO only) or presence of 80 mM Compound 1. Readings were taken from 25 to 99° C., with only the relevant portions of the curves shown. A single experiment (representative of three independent assays with two replicates each) is shown.

FIG. 17 shows Compound 1 inhibition of in vivo VirF activation in Shigella. β-galactosidase (LacZ) activity was assayed at the indicated concentrations of inhibitor Compound 1 from a virB-lacZ transcriptional fusion in Shigella, with native VirF expression. Activity in the absence of Compound 1 was set to 100% and corresponded to approximately 3,000 Miller Units. Shigella grown at 30° C. illustrates basal expression of virB-lacZ. Results are the average of three independent experiments with two replicates each.

FIGS. 18A-18D show Compound 1 inhibition of VirF regulated virulence gene expression in Shigella. qRT-PCR was used to determine the relative quantity (RQ) of expression of icsA (FIG. 18A), virB (FIG. 18B), icsB (FIG. 18C) and ipaB (FIG. 18D) in Shigella grown at 30° C. (without Compound 1) and at 37° C. (without or with 20 and 40 μM Compound 1). RNA levels were normalized to two constitutively expressed genes, gapA and rrsA. RNA levels were set to 1 in the absence of Compound 1. Shigella grown at 30° C. illustrates basal expression of each gene. Error bars represent the standard error of the mean calculated from three independent replicates. Significance of inhibitor-treated values was calculated using a non-parametric Mann-Whitney test, with P-values less than 0.05 considered significant (*). Results are the average of three independent experiments with one replicate each.

FIGS. 19A-19B shows Compound 1 inhibition of Shigella host cell invasion but not host cell metabolism. FIG. 19A shows the invasion index of Shigella into L-929 (mouse fibroblast) cells was determined by gentamycin protection assay. Cultures were grown in the absence or presence of Compound 1 (20 μM and 40 μM). A mxiH⁻ Shigella strain (SH116) and an ipgD⁻ strain (SME4331) grown at 30° C. were used as negative controls of invasion. As an additional control, 40 μM Compound 1 and Shigella grown at 37° C. without inhibitor were added at the same time to L-929 cells to test the effect of inhibitor on invasion by Shigella with a preformed T3SS and on host cells. The absorbance level in the absence of inhibitor and the presence of DMSO was set to 100%. Assays in (FIG. 19A) and (FIG. 19B) were performed in triplicate and error bars represent the standard error of the mean.

FIG. 20 reporter fusions used for in vivo assays. VirF-activated virB-lacZ fusion (Top) and LacI-repressed hts-lacZ control fusion (Bottom), each shown in their uninduced (−) IPTG state (left) and their induced (+) IPTG state (right). Gray rectangles: VirF and Lad (lacO) binding sites. Gray arrows: lacZ gene expressed from virB or synthetic hts promoter. Thick black/gray lines: Promoter region DNA. Right angle black arrows: Transcription start sites. Rounded gray shapes: RNAP, VirF or Lad proteins. Wavy lines: active transcription in the presence of IPTG.

FIG. 21 shows MBP-VirF protein after purification using amylose affinity chromatography. The image represents a single lane of a 12% SDS-PAGE gel stained with Coomassie Brilliant Blue. Numbers to the left of the gel indicate the positions of protein standards with the indicated molecular masses. MBP-VirF protein has a predicted molecular mass of 73 kDa.

FIG. 22 shows growth curves of E. coli strains in the absence or presence of Compound 1. Strains carrying virB-lacZ and VirF expressed from pHG165 (SME4382, squares) or hts-lacZ and Lad expressed from pHG165 (SME3359, circles) grown in the absence (−) or presence (+) of Compound 1 (44 μM), or the absence or presence of DMSO (0.3%). Results are the average of three replicates, error bars represent the standard error of the mean.

FIG. 23 shows relative quantification of mRNA levels of the constitutively expressed genes gapA and rrsA of Shigella. Levels of gapA were normalized to the rrsA values, and vice versa. Cultures were grown at 30° C. (Black bars) or at 37° C. with 40 μM Compound 1 (Grey bars). Values are relative to the mRNA levels of gapA (for gapA) or rrsA (for rrsA) for Shigella grown at 37° C. with no Compound 1, which were set to one. Error bars represent the standard error of the mean calculated from three independent replicates.

FIG. 24 shows growth curves of Shigella in the absence or presence of Compound 1. Shigella strain carrying ipgD⁻ (SME4331) in the absence (filled square) or presence of Compound 1 (20 μM, open squares; 40 μM, filled circles). Results are the average of three replicates, error bars represent the standard error of the mean.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention relates to compounds that can modulate protein members of the AraC family of bacterial transcriptional activators, and thereby the compound can be used to inhibit bacterial infections. The AraC proteins can be targets for the antibacterial agents, such as the compounds provided herein. The present invention includes in vivo high throughput screen to identify inhibitors (e.g., compounds of the invention) of the AraC family activator protein RhaS. The screen used two reporter fusions; one to identify potential RhaS inhibitors, and a second to eliminate non-specific inhibitors from consideration. One compound with excellent selectivity, OSSL_{—}051168 (i.e., Compound 1, also known as SE-1), was chosen for further study and to prepare analogs of, many of which analogs are substantially functional and many have similar effectiveness. Compound 1 inhibited in vivo transcription activation by the RhaS DNA-binding domain to the same extent as the full-length protein, indicating that this domain was the target of its inhibition. Growth curves showed that Compound 1 did not impact cell growth at the concentrations used in this study. In vitro DNA binding assays with purified protein suggest that Compound 1 inhibits DNA binding by RhaS. In addition, we found that it inhibits DNA binding by a second AraC family protein, RhaR, which shares 30% amino acid identity with RhaS. Compound 1 did not have a significant impact on DNA binding by the non-AraC family proteins CRP and Lad, suggesting that the inhibition is likely specific for RhaS, RhaR, and possibly additional AraC family activator proteins.

The present invention can overcome the ever-growing problem of bacterial antibiotic resistance, because the compounds function to inhibit the infection of cells rather than kill the bacteria. Traditionally, the molecular targets for antibacterial agents have been processes that are essential for bacterial growth. However, inhibition of such essential processes exerts substantial selective pressure for the emergence of resistance mechanisms that overcome the inhibition. One alternative strategy involves targeting virulence factors, where the compounds of the invention target virulence factors. The non-essential nature of virulence factors may reduce resistance development. Alternatively, the activator proteins that are required for the expression of bacterial virulence factors share the advantage of being non-essential, but tend to be much more conserved than virulence factors. Unlike the dramatic and long-term effects traditional antibiotics can have, targeting the expression of virulence factors with the compounds of the invention has the potential to be considerably less disruptive to the gut microbiota. Thus, the compounds of the invention can function as inhibitors of activator proteins that are required by bacterial pathogens for the expression of virulence factors and can be used as novel antibacterial agents.

The compounds of the invention can target proteins to modulate protein members of the large AraC family of transcriptional activators so as to inhibit activation of expression of genes involved in carbon metabolism, stress responses, or virulence. Indeed, large numbers of pathogenic bacteria, including many priority antibiotic resistant pathogens, require AraC family transcription activators for virulence factor expression and thereby to cause disease, and the compounds of the invention can be used against them. Many AraC family activators are required for the expression of multiple virulence factors; thus, blocking the function of the AraC family activator with the compounds of the present invention has the potential to substantially ameliorate virulence and disease.

The present invention provides small molecule inhibitors of AraC family virulence factor regulators. It is conceived that the compounds of the invention can inhibit a relatively diverse set of different AraC family activators. The compounds of the invention can result in very large reductions in virulence, and target AraC family activators for novel antibacterial agents.

The compounds of the invention can target AraC family activators RhaS and RhaR, which activate expression of the L-rhamnose catabolic operons in E. coli. RhaR activates expression of the operon that encodes RhaS and RhaR, and RhaS activates expression of the operons that encode the L-rhamnose catabolic enzymes and the L-rhamnose transport protein. Despite the fact that RhaS and RhaR appear to have arisen by gene duplication, and both activate transcription in response to the effector L-rhamnose, they share only 30% identity at the amino acid level.

The compounds of the invention can be used as AraC inhibitors for use as antibacterial agents. We used an in vivo high-throughput screen to identify the compounds that function as inhibitors of RhaS with the rationale that, similar to the hydroxybenzimidazole class of inhibitors, some might inhibit multiple AraC family activators. The in vivo screen circumvented the solubility problems that plague most AraC family activators, and had the further advantage that only compounds that were able to successfully enter Gram-negative bacterial cells would be identified. A secondary screen differentiated the desired RhaS inhibitors from non-specific inhibitors. The most potent of the inhibitors identified, Compound 1, was found to inhibit DNA binding by purified RhaS and RhaR proteins, but not by the unrelated CRP or Lad proteins. Compound 1 was subsequently analoged to provide analogs (e.g., compounds of the invention, such as Compound 2) that also have the same functionality. As can be seen, Compound 1 and Compound 2 are analogs of each other, and thereby other analogs that modify the alkyl group are likely candidates. Also, derivatives, prodrugs, and pharmaceutically acceptable salts of Compound 1 and Compound 2 can be used in the present invention. Additionally, the present invention can include pharmaceutical compositions having Compound 1 and/or Compound 2 and a pharmaceutically acceptable carrier and/or excipient. The compositions can include Compound 1 and/or Compound 2 in a therapeutically effective amount for inhibiting or treating a microbial infection, or inhibition of a pathway thereof, or inhibition of a protein and/or protein function in a microbial infection, in accordance with the descriptions provided herein.

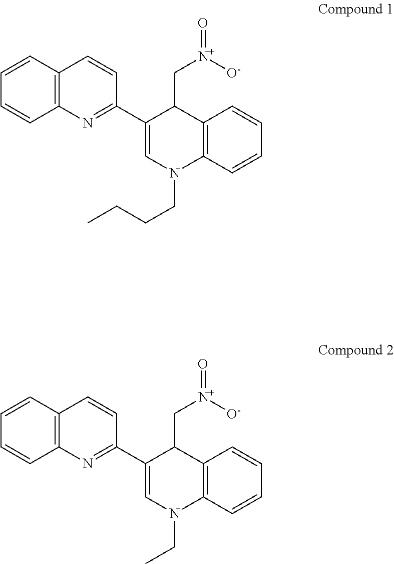 [SMILES: CCCCN1C=C(c2ccc3ccccc3n2)C(C[N+](=O)[O-])c2ccccc21 ; CCN1C=C(c2ccc3ccccc3n2)C(C[N+](=O)[O-])c2ccccc21]

Additionally, the compounds of the invention can include the structure of Formula 1 or Formula 2, or derivatives, prodrugs, and/or pharmaceutically acceptable salts.

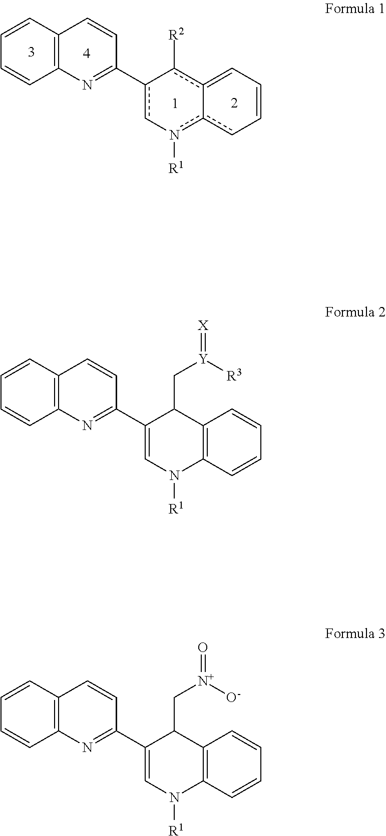

In Formula 1 and Formula 2 and Formula 3, R¹ or R² or R³ can be independently any substituent. As such, R¹ can be a hydrogen, halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, derivatives thereof, substituted or unsubstituted, or combinations thereof as well as other well-known chemical substituents. R¹ or R² or R³ can be independently selected from the group of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano(—C≡N), isocyano (—N⁺≡C⁻), cyanato (—O—C≡N), isocyanato (—O—N⁺≡C⁻), isothiocyanato (—S—C≡N), azido (—N═N⁺═N⁺), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₆-C₂₀ arylamido (—NH—(CO)-aryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—S₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O⁻)), phospho (—PO₂), phosphino (—PH₂), derivatives thereof, and combinations thereof. The alkyl groups of these substituents can be short, such as C₁-C₁₂, C₁-C₁₁, C₁-C₁₀, C₁-C₉, C₁-C₈, C₁-C₇, C₁-C₆, C₁-C₅, C₁-C₄, C₁-C₃, or C₁-C₂, straight or branched and/or substituted or unsubstituted. For example R¹ can be methyl, ethyl, propyl, isopropyl, butyl, tertbutyl, pentyl, hexyl, cyclohexyl, benzyl, heptyl, and any configuration thereof, substituted or unsubstituted. X can be S, O, N, or P. Y can be C or N. The dashed lines illustrate optional bonding where the nitrogen of ring 1 may be aromatic or aliphatic, where Ring 1 can have at least one double bond and the N of Ring 1 can include 3 or 4 bonds, when having 4 bonds an anionic counter ion can be present. The anionic counter ion can be a halogen, or any other counter ion or it can be a salt. Ring 2 can include 2 or 3 double bounds, where an aromatic Ring 2 is preferred.

Pharmaceutical compositions can include the compounds of the invention, and can include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (e.g., Tween®), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes. Such compositions should contain a therapeutically effective amount of the compound, together with a suitable amount of carrier so as to provide the form for direct administration to the patient.

The compositions described herein can be administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration. Common carriers or excipients can be used for preparing pharmaceutical compositions designed for such routes of administration.

The compounds of the invention can be used to inhibit AraC-family activators Rns and VirF, which are required for the expression of key virulence factors in Enterotoxigenic E. coli [ETEC] and Shigella, respectively. Both ETEC and Shigella show rapid development of resistance to available antibiotics. We have identified and investigated the inhibitory mechanism of a small molecule inhibitor, Compound 1, of Rns and VirF. This inhibitor can be an antibacterial agent targeting ETEC and Shigella—pathogens responsible for an immense global human disease burden. The compounds of the invention can cause inhibition of transcription activation by Rns and VirF, and the compounds will prevent the expression of genes that encode critical virulence factors in ETEC and Shigella, and thereby reduce the ability of these pathogens to cause human disease. Rns is required for the expression of most, if not all, of the colonization factor antigens [CFAs] that ETEC uses for host cell attachment and efficient toxin delivery. Loss of CFA expression eliminates infection and diarrhea. VirF is the master regulator of virulence gene expression in Shigella, and loss of VirF eliminates host cell invasion as well as disease and pathology. Thus, the compounds that are effective inhibitors of Rns and VirF can be antibacterial agents that block disease caused by these human pathogens.

The compounds of the invention can target well-conserved activator proteins that are required for bacterial virulence factor gene expression. The compounds of the invention inhibit AraC-family activators that are essential for virulence factor expression in many human pathogens, including those that cause diarrheal disease [pathogenic E. coli and Shigella—subjects of this work—are Category B priority biodefense pathogens].

In one embodiment, the compounds of the invention can inhibit or reduce or treat diarrhea caused from bacterial infections. This can be by targeting and inhibiting AraC-family activators that are required for virulence factor expression in many diarrheal pathogens. The compounds of the invention can be inhibitors of the AraC-family virulence regulators Rns and VirF from ETEC and Shigella in order to inhibit or treat diarrhea.

The compounds of the invention can be AraC-family inhibitors. We previously used high-throughput screening to identify inhibitors of the AraC-family activator RhaS, since its molecular mechanisms are well characterized. The most potent inhibitor is Compound 1, which blocks DNA binding by both purified RhaS protein, and the isolated DNA-binding domain of RhaS. Two computational analyses [SwissDock and BSP-SLIM] predict that Compound 1 docks within a conserved pocket between the two HTH motifs of various AraC-family DNA-binding domain structures, which suggests that the inhibitor sterically interferences with DNA binding. The conservation of the pocket structure suggested that Compound 1 might inhibit DNA binding by other AraC-family proteins, and Compound 1 also inhibits DNA binding by RhaR and by the virulence regulators Rns [ETEC] and VirF. It is expected that the compounds of the invention can also have such activity and functionality.

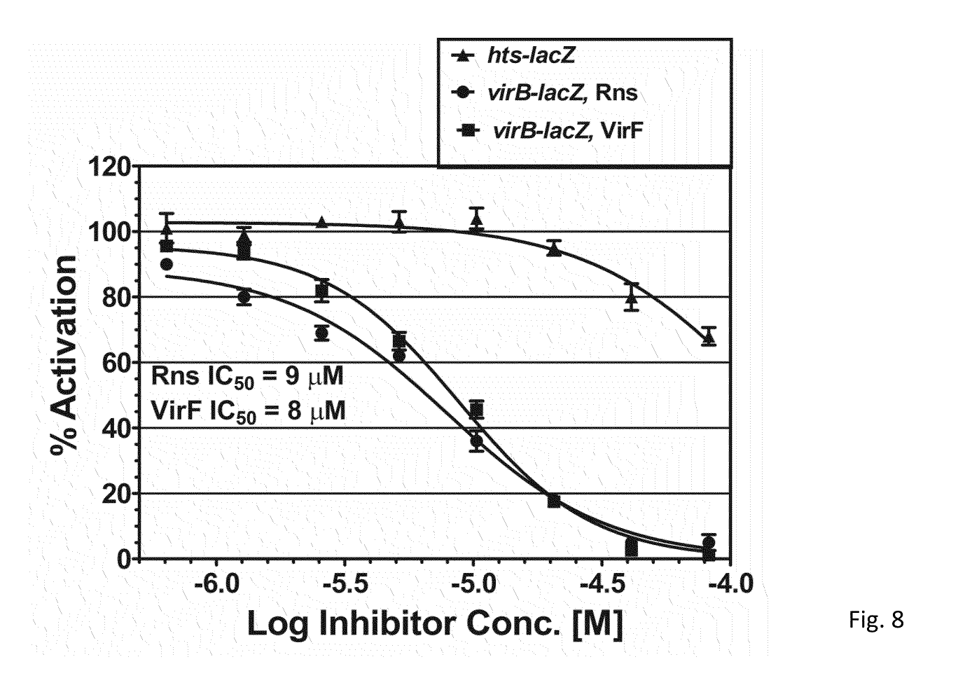

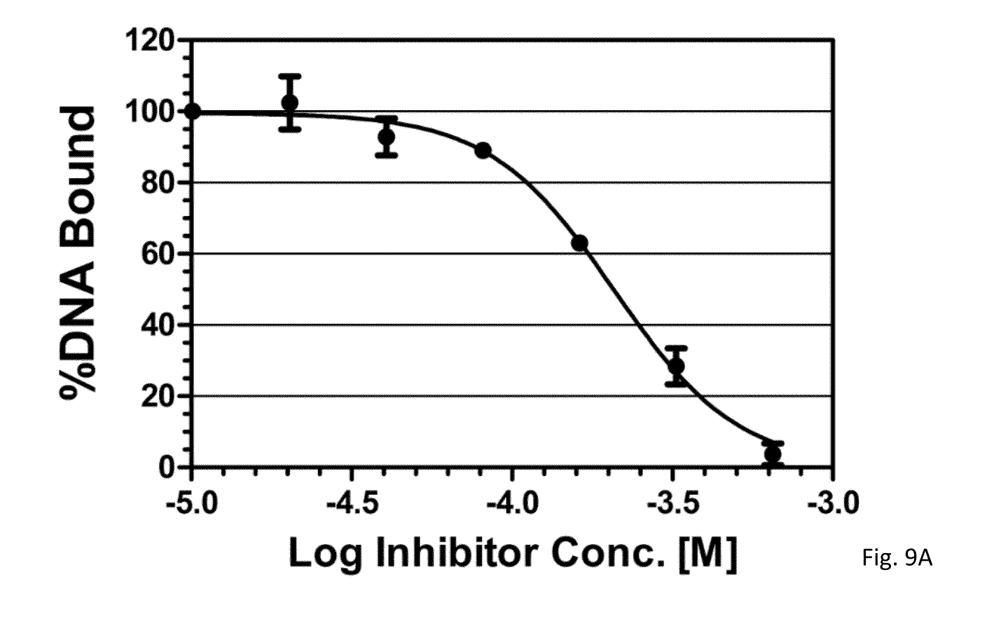

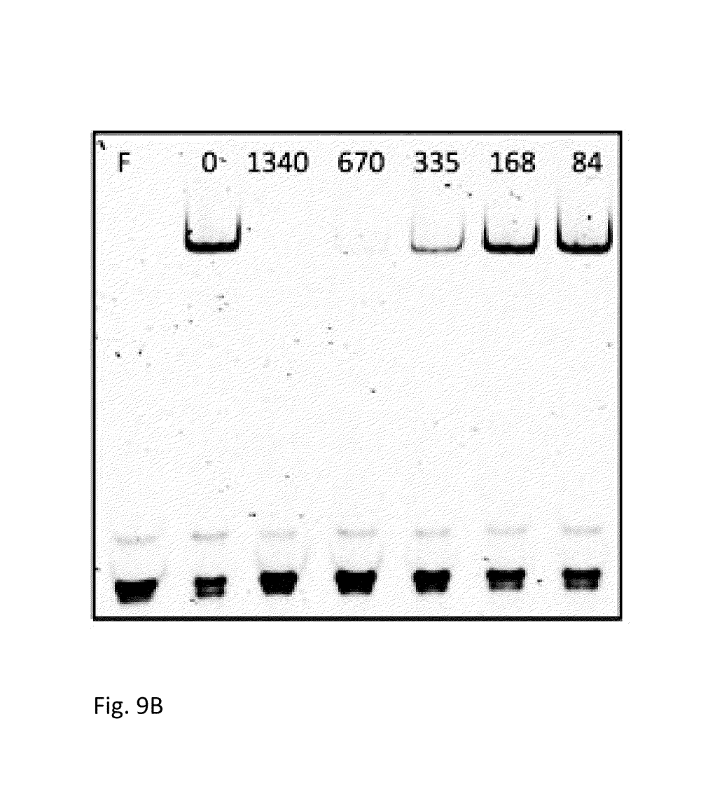

The compounds of the invention can be used for inhibition of virulence in vivo and in vitro activation by Rns and VirF. Compound 1 inhibits in vivo activation by Rns and VirF to a similar, if not greater, extent than full-length RhaS [FIG. 8]. Compound 1 has also been found to inhibit DNA binding by purified RhaS and RhaS-DBD. Compound 1 similarly inhibits in vitro DNA binding by purified Rns [FIG. 9A] and VirF [FIG. 9B], but not the unrelated activators CRP and LacI. Rns and VirF proteins were purified as Maltose binding protein [MBP] fusions, which previous studies found were fully active and exhibited greatly increased solubility.

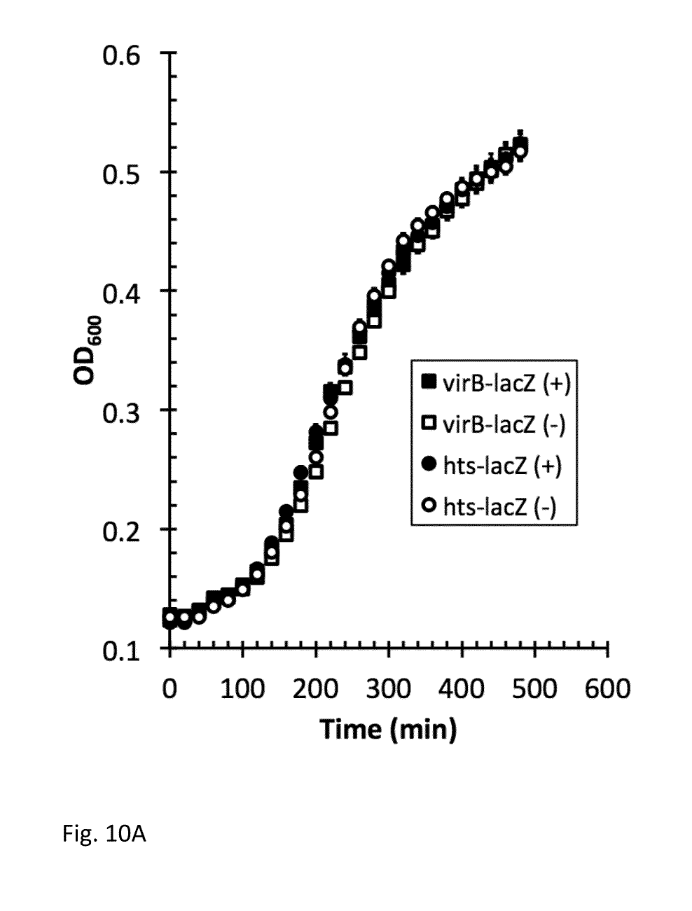

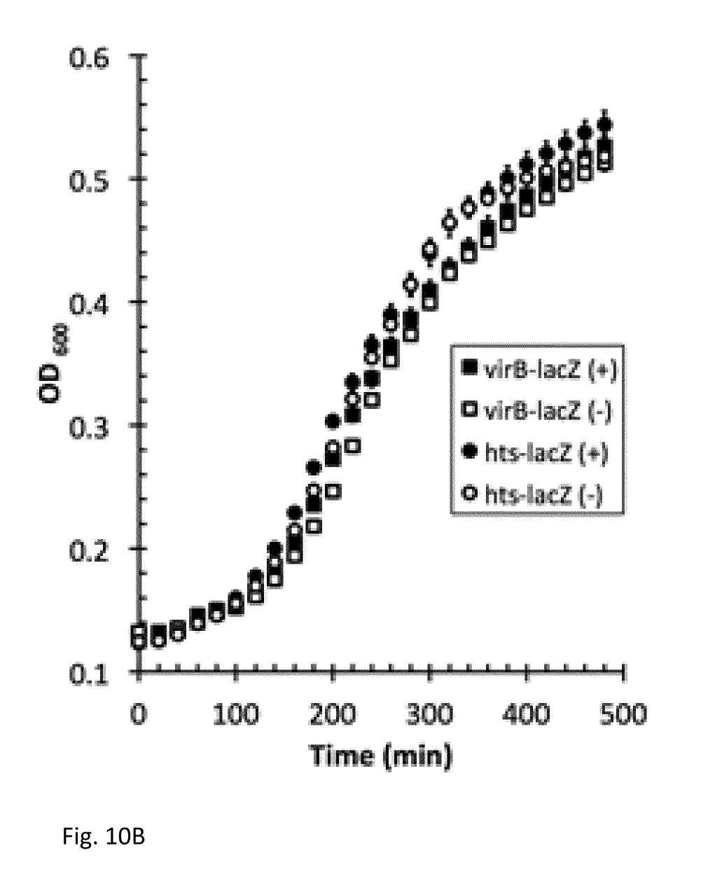

It was found that Compound 1 does not inhibit growth of the E. coli strains used for the in vivo studies. We assayed the growth of the strains carrying the VirF/Rns-activated and the LacI-repressed reporter fusions, grown in the same minimal medium used for the in vivo dose response assays. We found that there was no impact of the inhibitor on the growth of the cells for either strain [FIGS. 10A and 10B].

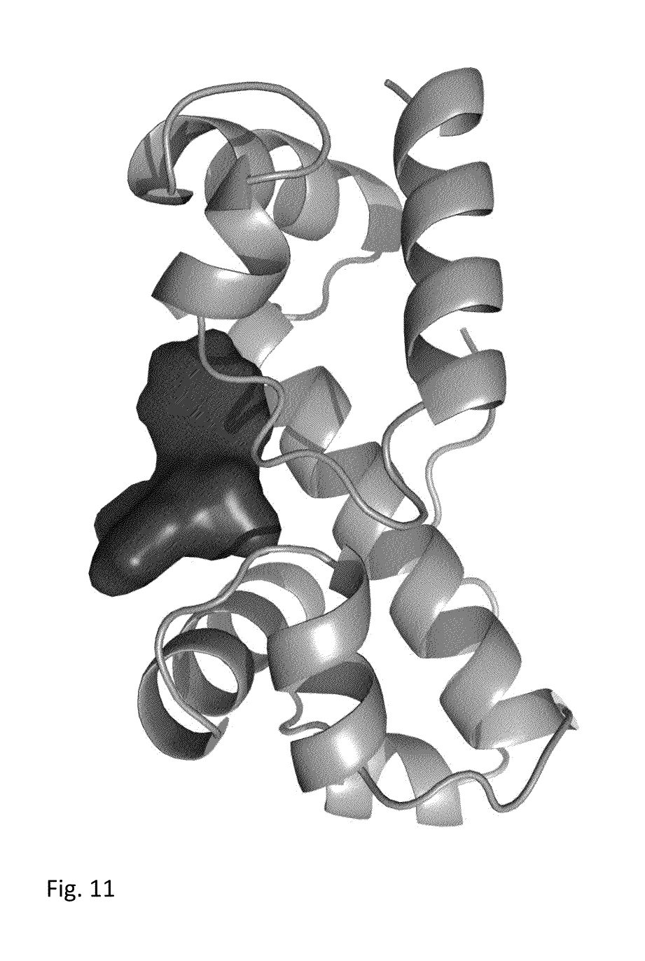

We used SwissDock and BSP-SLIM to computationally predict the docking site of Compound 1 with the AraC family protein MarA [DNA binding domain only, pdb BL01]. The most energetically favorable poses in both cases placed Compound 1 in approximately the same position—in a conserved pocket between the two HTH motifs [FIG. 11]. The same position was also found when Compound 1 was docked with the structure of the full-length ToxT protein [pdb 3 GBG] and with a computational model [predicted using I-TASSER] of full-length RhaS. This pocket is universally conserved among AraC-family DNA binding domain structures to date [including several recently deposited but unpublished structures], which suggests that Compound 1, as well as the analogs thereof, might broadly inhibit DNA binding by AraC-family proteins.

We assayed the effect of Compound 1 on the interaction of Shigella with cultured human epithelial cells. Shigella flexneri strain M90T was grown to maximize type III secretion system [Shigella] expression. Shigella was assayed for invasion of HeLa cells. The bacterial cells were grown in the presence of Compound 1 prior to addition to tissue culture wells. Invasion assays involved, briefly, incubation of Shigella cells in tissue culture wells, washing to remove non-adherent bacteria, addition of gentamycin to kill bacterial cells that had not invaded the epithelial cells, trypsin treatment to dislodge epithelial cells, serial dilutions and plating on LB to enumerate bacterial colonies

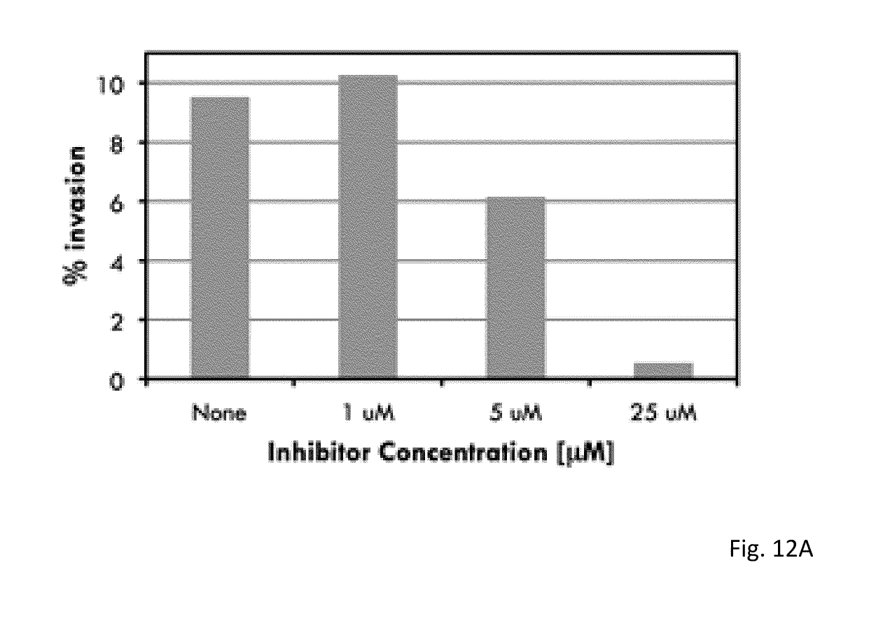

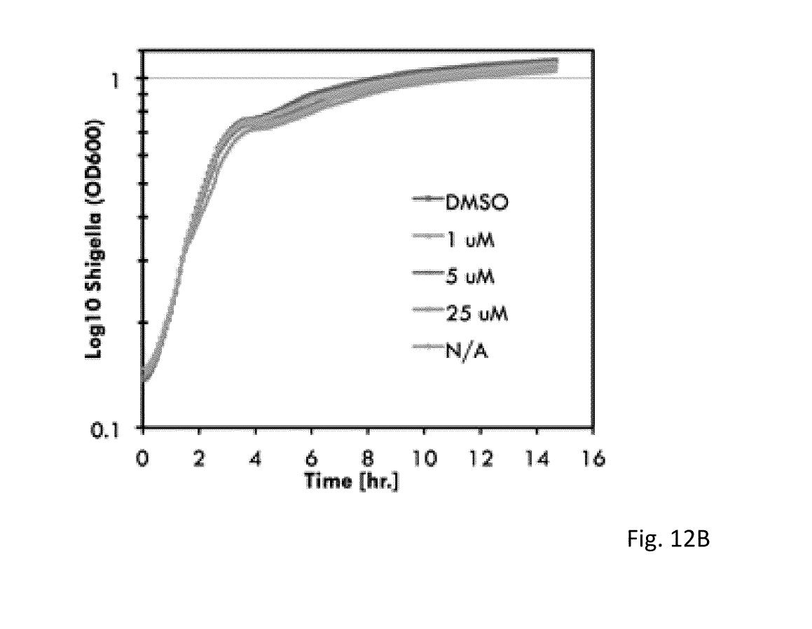

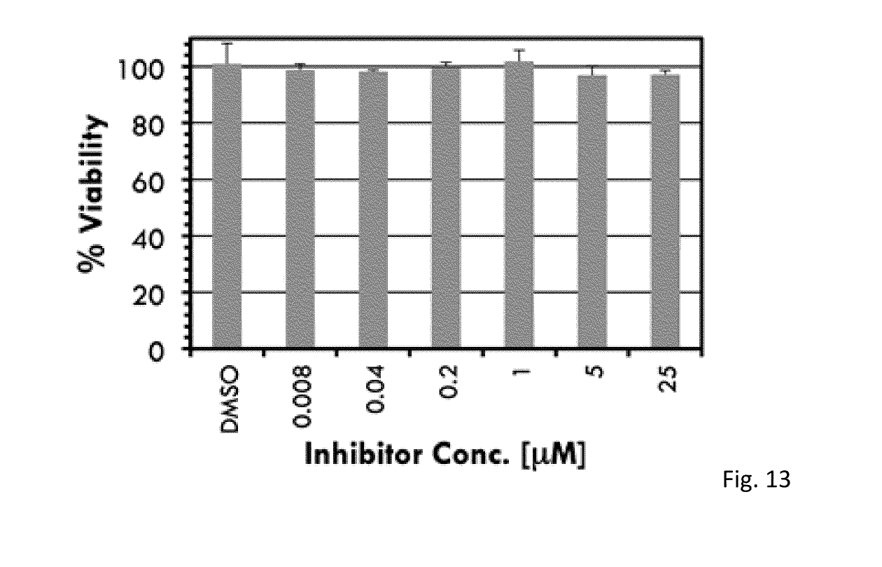

We found that Compound 1 reduced the ability of Shigella to invade human epithelial cells but not the Shigella growth rate [FIGS. 12A-12B]. We expect that Compound 1 can impact host cell interaction but have little or no effect on growth rate of Shigella, based on our results with non-pathogenic E. coli strains. We also showed using Alamar Blue [resazurin] metabolic activity assays [Life Technologies, Grand Island, N.Y.] that Compound 1 did not have any effect on the metabolic activity of the tissue culture cells used in the invasion assays [FIG. 13].

VirF is an AraC family transcriptional activator that is required for the expression of virulence genes associated with invasion and cell-to-cell spread of S. flexneri, including multiple components of the type three secretion system (T3SS) machinery and effectors. We tested a small molecule compound, Compound 1, which we had identified as an effective inhibitor of the AraC family proteins RhaS and RhaR, for its ability to inhibit VirF. Cell-based reporter gene assays in E. coli and Shigella, as well as in vitro DNA binding assays with purified VirF demonstrated that Compound 1 inhibited DNA binding and transcription activation by VirF. Analysis of mRNA levels using qRT-PCR further demonstrated that Compound 1 reduced the expression of the VirF-dependent virulence genes icsA, virB, icsB and ipaB in Shigella. We also performed eukaryotic cell invasion assays and found that Compound 1 reduced invasion by Shigella. The effect of Compound 1 on invasion required pre-incubation of Shigella with Compound 1, consistent with the hypothesis that Compound 1 inhibited expression of VirF-activated genes required for formation of the T3SS apparatus and invasion. We found that the same concentrations of Compound 1 did not have any detectable effects on the growth or metabolism of the bacterial or eukaryotic host cells, respectively, indicating that the inhibition of invasion was not due to general toxicity. Overall, Compound 1 inhibits transcription activation by VirF, exhibits selectivity toward AraC family proteins. AraC family proteins activate virulence gene expression in many pathogenic bacteria including Shigella flexneri (VirF), Vibrio cholerae (ToxT), Enterotoxigenic Escherichia coli (ETEC, Rns/CfaD), and Pseudomonas aeruginosa (ExsA), and thereby the compounds of the invention can be used for inhibiting or treating infections of the same.

In one embodiment, the compounds of the present invention can inhibit VirF activation of expression of a cascade of genes responsible for the formation of the type 3 secretion system (T3SS) machinery, invasion of Shigella into host epithelial cells, and cell-to-cell spread. The compounds can inhibit VirF and thereby inhibit activation of the expression of the icsA and virB virulence genes. The compounds can inhibit expression of the icsA gene that encodes the IcsA/VirG protein, which aids intracellular movement of the pathogen by mediating actin-based motility, and thereby inhibit the same. The compounds can inhibit the expression of the virB gene that encodes a transcriptional activator, VirB, which in turn activates the expression of many virulence-associated genes (including the mxi, spa and ipa operons), and thereby inhibit the same. The compounds can inhibit the expression of the genes in the ipa operon that encode effector proteins (IcsB, IpaA, IpaB, IpaC and IpaD) which translocate directly into host cells, and thereby inhibit the same. Among the effectors, IpaB, has major roles in formation of pores in the host cell membrane, lysis of phagosomes and macrophage apoptosis, and thereby the compounds can inhibit such pore formation and inhibit lysis of phagosomes and inhibit macrophage apoptosis. Another effector, IcsB, prevents the triggering of host autophagy, a process that can be used by host cells to export invading bacteria to lysosomes for degradation, and thereby the compounds can inhibit the prevention of triggering host autophagy. Given that VirF is required for VirB expression and that VirB activates the expression of multiple virulence-associated genes that are required for the earliest steps in the successful invasion of host cells by Shigella, VirF is considered the master regulator of Shigella virulence, and inhibition of VirF can inhibit corresponding disease sates.

It has been found that Compound 1 can inhibit the master Shigella virulence activator, VirF of the AraC family activators. Our results showed that the Compound 1 effectively inhibited VirF DNA binding and therefore its ability to activate transcription in vivo. Analysis of mRNA levels for direct and indirect targets of VirF showed a significant reduction in the expression of these genes in the presence of Compound 1. Compound 1 also demonstrated significant inhibition of Shigella invasion of epithelial cells in tissue culture without showing any detrimental effects on the growth of the bacterial cells or the metabolism of the host cells. Reductions in gene expression and invasion are due to Compound 1 inhibition of VirF activity. Compound 1 inhibits transcription activation by the Shigella master virulence regulator, VirF.

The subjects that can be treated with the compounds of the invention can be any animal, where humans are an example. However, any animal having or susceptible to a microbial infection or that has been exposed to a microbial infection may be the subject.

The compounds can inhibit the microbe from entering into a cell. The cell can be any type of cell of the subject. Examples of such cells can be those that are susceptible to invasion by the microbes, such as the microbes described herein.

The inhibition or treatment provided by the compounds of the invention can be compared to the absence of the presence or administration of the compounds of the invention.

### Experimental 1

Bacterial Growth Media and Conditions.

Cultures for the primary high-throughput screen were grown in tryptone broth plus ampicillin (TB; 0.8% Difco tryptone, 0.5% NaCl, pH 7.0; all % recipes are w/v except glycerol and DMSO, which are v/v). Cultures for subsequent in vivo assays were grown in MOPS [3-(N-morpholino)propanesulfonic acid]-buffered minimal medium with 0.4% glycerol as the carbon source, and supplemented with 0.2% Difco Casamino acids, 0.002% thiamine and ampicillin. Cultures for phage P1 vir infection were grown in tryptone-yeast extract broth (TY; 0.8% Difco tryptone, 0.5% Difco yeast extract, 0.5% NaCl, pH 7.0) supplemented with 5 mM CaCl₂. Difco Nutrient Agar was used routinely to grow cells on solid medium. Difco MacConkey Base Agar supplemented with 1% sorbitol or maltose was used to screen for sorbitol- and maltose-deficient phenotypes. Minimal sorbitol plates contained 1×E salts, 0.2% sorbitol, 0.002% thiamine, 1.25 mM Na₄P₂O₇ and 1.5% Bacto agar. Ampicillin (200 μg/mL), tetracycline (20 μg/mL), chloramphenicol (30 μg/mL), gentamycin (20 μg/mL), L-rhamnose (0.2%), glucose (0.2%), and isopropyl-β-D-thiogalactopyranoside (IPTG; 0.1 mM unless otherwise noted) were added as indicated. All cultures were grown at 37° C. with aeration, unless otherwise noted.

High-Throughput Screening Compound Library.

High-throughput screening was performed using the compound library at the University of Kansas High Throughput Screening Laboratory, which consisted of approximately 110,000 compounds. Compounds were purchased from ChemBridge Corp. (San Diego, Calif.), Chemdiv, Inc. (San Diego, Calif.), Prestwick Chemicals (Illkirch, France) and MicroSource Discovery Systems, Inc. (Gaylordsville, Conn.). Compounds were selected based on structural diversity and drug-like properties.

Primary High-Throughput Screen.

An overnight culture of SME3006 grown in TB with ampicillin was diluted 1:100 into fresh TB with ampicillin that had been pre-warmed to 37° C. Cells were grown to an OD₆₀₀ of 0.1 and growth was stopped on ice for approximately 30 min. Using a Multidrop 384 (Thermo Scientific, Hudson, N.H.), 35 μL of this cell culture was added to each well of a 384-well plate (Nunc, Rochester, N.Y.). In addition to cells, each well in column 1 of the plate contained 20 μL 2.5% dimethyl sulfoxide (DMSO) and 10 μL water (uninduced control); each well in column 2 contained 20 μL 2.5% DMSO and 10 μL 2% L-rhamnose (induced control); and each well in columns 3-24 contained 20 μL of a library compound at 25 μg/mL in 2.5% DMSO and 10 μL 2% L-rhamnose. Plates were incubated statically for 3 hr. at room temperature to allow rhaB-lacZ induction, followed by addition of 25 μL lysis/ONPG (o-nitrophenyl-β-D-galactopyranoside) buffer [3 parts ZOB⁴ to 1 part 10 mg/mL lysozyme (Sigma, St. Louis, Mo.) dissolved in PopCulture cell lysis reagent (EMD Chemicals, Inc., Gibbstown, N.J.)]. After approximately 3 hr. of incubation at room temperature, OD₄₀₅ readings were taken for each well using an EnVision Multilabel Reader (PerkinElmer, Waltham, Mass.).

The average of the 16 induced and 16 uninduced wells on each individual plate were used to calculate the percent activation for each well as follows.

$$\% activation = \frac{{OD}_{405} - average {OD}_{405} of uninduced controls}{\begin{matrix}average {OD}_{405} of induced controls - \\ average {OD}_{405} of nuinduced controls\end{matrix}}$$

Z-factors were calculated for each individual 384-well plate screened.

HTS Strain Construction.

The tester strain for the primary high-throughput screen was SME3006. This strain carried the RhaS-activated Φ(rhaB-lacZ)Δ84 fusion in single copy on the chromosome. The rhaBAD promoter in this fusion includes the full binding site for the RhaS protein, but not the upstream binding site for CRP. This ensures that RhaS is the sole activator of this fusion, and that inhibition of CRP protein activity would not decrease LacZ expression. This strain also carries ArhaS and recA::cat on the chromosome and RhaS-expressing pHG165rhaS, which modestly increases rhaB-lacZ expression levels compared with chromosomal rhaS expression.

The control strain for the secondary high-throughput screen and subsequent experiments was SME3359, and carries the LacI-repressed fusion and LacI-expressing pHG165lacI. The fusion consists of lacZ under the control of an artificial promoter (Phts) with an induced expression level similar to that of the induced rhaBAD operon. Phts is regulated by Lad and induced with IPTG. The Phts core promoter elements include a near-consensus −35 sequence and a −10 sequence followed by a lacO₁ operator sequence that overlaps the transcription start site with the same spacing as lacO₁ at lacZYA. To construct the upstream half of Phts, oligo 2829 was annealed to oligo 2788 and the primers were extended using the Expand High Fidelity PCR System (Roche Applied Science, Indianapolis, Ind.). The downstream half of the Phts promoter was constructed by similarly annealing and extending oligos 2789 and 2790.

The PCR products were cleaned up with a QIAquick PCR Purification Kit (Qiagen, Inc., Valencia, Calif.), digested with Earl, EcoRI, and BamHI (New England Biolabs, Ipswich, Mass.), and then ligated to EcoRI- and BamHI-digested pRS414. The sequence of both DNA strands of the cloned region was confirmed (Northwestern University Genomics Core, Chicago, Ill.). Phts-lacZ was recombined onto kRS45 and integrated as a single-copy lysogen into the chromosome of SME1085. Likely single-copy lysogens were identified by β-galactosidase assay and confirmed by PCR. The resulting strain was transformed with LacI-expressing pHG165lacI.

Secondary High-Throughput Screen.

We re-screened the top ˜5% most inhibitory compounds from the primary screen. The secondary screen was performed essentially as the primary screen, except for the following changes: Cells were grown in MOPS-buffered minimal medium with ampicillin rather than TB with ampicillin. Compounds were tested against both SME3006 and SME3359. For plates containing SME3359, 6.5 mM IPTG was used as the inducer rather than 2% L-rhamnose, and the first and second columns were uninduced and induced controls, analogous to above.

Strain Construction for In Vivo Dose-Response Studies.

For dose-response studies, a rhaB-lacZ reporter strain was designed that allowed IPTG induction of RhaS or RhaS(163-278) expression from pHG165 (and thus rhaB-lacZ expression). The strain was constructed by introducing malP::lacIq into SME3000 via phage P1vir-mediated generalized transduction. First, SME3000 was transduced with Δcrp-3 zhc-511::Tn10 from SME1403, selecting for tetracycline resistance and screening for sorbitol deficiency (indicating Δcrp) using MacConkey sorbitol. SME3000 Acrp-3 zhc-511::Tn10 was transduced with crp⁺ malP::lacI^{q} from SG22166, selecting for growth on minimal sorbitol and screening for maltose deficiency (indicating malP::lacI^{q}) using MacConkey maltose. Finally, SME3000 malP::lacI^{q} zhc-511::Tn10 was transduced with recA::cat from SME1048 by selecting for chloramphenicol resistance, to make strain SME3632.

In Vivo Dose-Response Experiments.

The top hit identified in the screen was 1-ethyl-4-(nitromethyl)-3-quinolin-2-yl-4H-quinoline, Compound 2. The closely related compound, Compound 1,1-butyl-4-(nitromethyl)-3-quinolin-2-yl-4H-quinoline, was obtained from eMolecules, Inc. (Solana Beach, Calif.; catalog #3761-0013) or Princeton BioMolecular Research (Princeton, N.J.; catalog #OSSL_{—}051168), with the Princeton named used here. Compound 1 was dissolved in 100% DMSO and then the solution was further diluted in 100% DMSO in a 2-fold series for a total of seven concentrations. Dose-response assays were performed in 96-well plates with one column each of uninduced and induced controls for each strain, and one column with a concentration curve of Compound 1 for each strain. Uninduced wells contained 20 μL water and 40 μL 10% DMSO; induced wells contained 20 μL induction solution (2% L-rhamnose and 6.5 mM IPTG) and 40 μL 10% DMSO; concentration curve wells contained 20 μL induction solution, 36 μL water and 4 μA diluted Compound 2. The appropriate cell culture (70 μL, grown to OD₆₀₀=0.1 in MOPS-buffered minimal medium with ampicillin) was added to each well and induced for 3 hr. at 37° C. Lysis/ONPG buffer (50 μL, as in Primary Screen) was added to each well and the plate was immediately placed into a PowerWave XS plate reader (BioTek Instruments, Inc., Winooski, Vt.), set to read the OD₄₂₀ of each well every 15 min for 4 hr. A single time point within the linear range of β-galactosidase activity was chosen for analysis for each strain. Due to differences in kinetics of lacZ reporter expression, the rhaB-lacZ strains were analyzed at 4 hours and the hts-lacZ strain was analyzed at 1 hr. Percent activity was calculated for each condition as described above, using the single time point with appropriate OD₄₂₀ levels for each strain. Error bars represent the standard error of the mean. IC₅₀ and maximal inhibition values were calculated using the XLfit add-in for Microsoft Excel (ID Business Solutions, Guildford, UK), and graphs were drawn using Prism (GraphPad, La Jolla, Calif.).

Growth Curves.

Strains SME3634 and SME3359 were grown overnight (˜16 hr) at 37° C. in MOPS-buffered minimal medium. Overnight grown cultures were diluted into the same medium plus rhamnose to an O.D₆₀₀ of 0.1, and IPTG was added. 1 mL aliquots of the cultures were added to wells of a 24-well plate, with the addition of either 44 μM Compound 1 (dissolved in 100% DMSO) or an equal volume of 100% DMSO. Growth was monitored at 20 min intervals for approximately 8 hr. at 37° C. with continuous shaking in a PowerWave XS plate reader (BioTek Instruments, Inc., Winooski, Vt.). Error bars represent the standard error of the mean.

Protein Purification.

All proteins for this study were expressed in E. coli. Untagged CRP was purified by Ni²⁺-affinity chromatography as described. Lad protein was purified by ammonium sulfate precipitation and phosphocellulose column chromatography as described. RhaS-GB1^{201b} and GB1^{b}-RhaR were purified by Ni²⁺-affinity chromatography. Briefly, plasmids derived from pET21 and expressing RhaS-GB1^{201b} and GB1^{b}-RhaR were transformed into competent cells of strains Acella™ (MoBiTec, Gottingen, Germany) or ArcticExpress(DE3) (Agilent Technologies, Santa Clara Calif.), respectively. The cells were grown in 1 liter TY plus ampicillin and rhamnose. Gentamycin was also added for GB1^{b}-RhaR. The cells were grown to OD₆₀₀ of 0.5, transferred to a 15° C. shaker, 0.1 mM IPTG was added, and then incubated overnight. Cells were harvested by centrifugation and then resuspended in 30 mL of cold binding buffer (20 mM Tris, 500 mM NaCl, 5 mM imidazole, pH 7.9) plus L-rhamnose. Cells were lysed by three cycles of freeze thaw [with addition of lysozyme (0.4 mg/mL), tris(2-carboxyethyl)phosphine (TCEP, 1 mM) and phenylmethylsulfonyl fluoride (PMSF, 1 mM), at −80° C.] followed by sonication, and then centrifuged to remove cell debris. The supernatant was applied using an AKTAexplorer FPLC (GE Healthcare) to a 5 mL HiTrap Chelating HP column (GE Healthcare) that had been charged with 50 mM NiSO₄, and equilibrated with 15 mL H₂O and then 15 mL binding buffer. After loading, the column was washed with 25 mL binding buffer, then 25 mL wash buffer (binding buffer, but with 60 mM imidazole) plus L-rhamnose. A 10 mL gradient of binding buffer with 60 mM to 250 mM imidazole from was run and then 15 mL of elution buffer (binding buffer, but with 250 mM imidazole). The ArcticExpress cold-adapted chaperonins Cpn10 and Cpn60 (14 monomers per unit) co-purified with GB1^{b}-RhaR, thus GB1^{b}-RhaR represented only approximately 20% of the total protein used in the assays.

In Vitro DNA Binding Assays.

Electrophoretic mobility shift assays were performed as described, with the following modifications. Reaction volumes were 12 μL total (with 5 μL loaded in each lane), in 1×EMSA buffer [10 mM Tris-HCl (pH 7.4), 1 mM KEDTA, 50 mM KCl, 1 mM dithiothreitol, 5% (v/v) glycerol, 0.1 mg/mL bovine serum albumin (BSA) and 500 ng salmon sperm DNA]. Reactions also contained additives as follows: RhaS-GB1^{201b}, L-rhamnose; GB1^{b}-RhaR, Nonidet P40 and L-rhamnose; CRP, cAMP; LacI, none. Purified proteins were buffer exchanged into 1×EMSA buffer minus BSA and salmon sperm DNA, and without the addition of additives. Electrophoresis was performed in 0.25×TBE (final concentrations: 22.25 mM Tris base, 22.25 mM boric acid, 500 μM disodium EDTA, pH 8.3). All EMSA reactions, including those without inhibitor, had a final concentration of 10% DMSO (OSSL_{—}051168 solvent). DNA probes were generated by hybridizing the following oligonucleotides (oligos): For RhaS-GB1^{201b}, oligo 3058 and oligo 3288; for GB1^{b}-RhaR, oligo 3056 and oligo 3287; for CRP, oligo 3161 and oligo 3162; and for LacI, oligo IR O1-For and oligo O1-Rev.

IRD700- and DY682-labeled oligos were from Eurofins MWG Operon. For each oligo pair, 100 μmol of each oligo was combined and the reaction was diluted in STE buffer (50 mM NaCl, 10 mM Tris-HCl, 1 mM EDTA, pH 8.0) to 20 μL, heated to 94° C. for 2 min, and cooled to room temperature. The double-stranded DNA probes were further diluted in STE, and 0.3-1 μL added to EMSA reactions. EMSA gels were imaged using an Odyssey infrared imager (LI-COR, Lincoln, Nebr.), and quantified using the Odyssey software, version 3.0.30. Error bars represent the standard error of the mean. Inhibition values were calculated and graphs were drawn as for in vivo dose-response experiments.

High-Throughput Screen to Identify Inhibitors of AraC Family Activators.

Our goal was to use high throughput screening to identify novel inhibitors that could block the function of AraC family activator proteins, and then create analogs thereof. Ultimately, such inhibitors have the potential to be developed into antibacterial agents that block virulence factor expression in human pathogens. Although it does not regulate virulence factor expression in any human pathogens, we chose the RhaS protein as our initial target since the molecular mechanisms used by RhaS to activate transcription are well characterized.

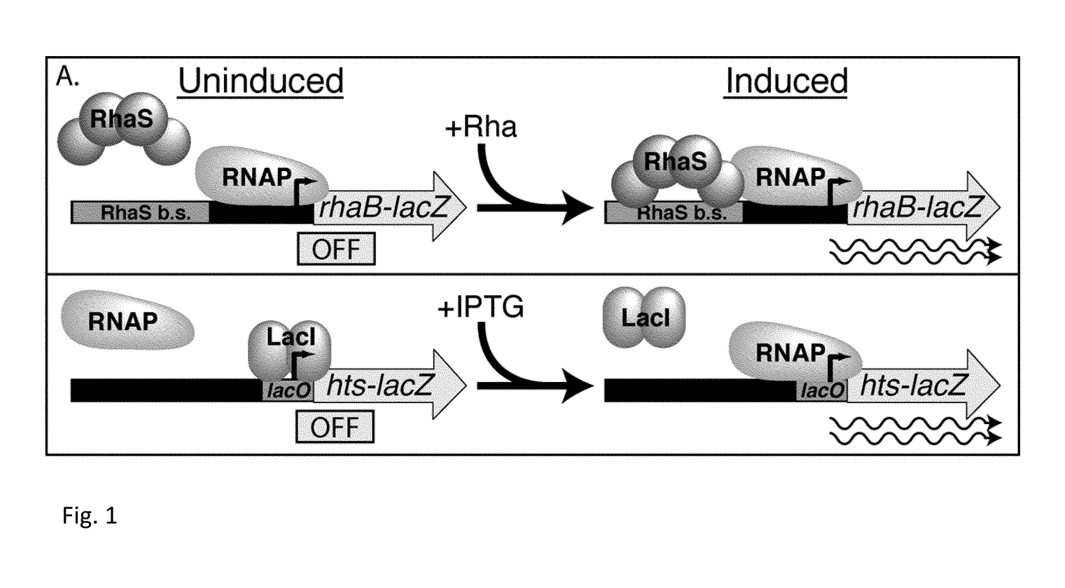

The first step of our in vivo high-throughput screen for RhaS inhibitors was to screen for compounds that decreased expression of a RhaS-activated rhaB-lacZ reporter fusion in whole cells (FIG. 1, top). Screening in whole cells (rather than with purified protein) was advantageous for two reasons. First, RhaS is very insoluble, and purified, active RhaS protein was not available at the time of this screen. Second, whole-cell screens are expected to identify only compounds that can enter, and remain active in the bacterial cell. The disadvantage of our whole-cell assay was that many compounds were expected to affect RhaS-activated LacZ reporter activity without directly affecting activation by RhaS. In order to screen out the vast majority of such indirect effects, each compound that reduced the RhaS-activated LacZ reporter activity in the primary screen was re-tested, both on the primary screening strain and on a secondary screening strain. The secondary screening strain was an isogenic control strain carrying a lacZ reporter fusion (hts-lacZ), with a synthetic promoter that was repressed by Lad, and did not require RhaS for activation (FIG. 1, bottom). Non-specific inhibitors that blocked β-galactosidase enzyme activity or cell growth, for example, were expected to decrease lacZ expression from both rhaB-lacZ and hts-lacZ. In contrast, the compounds of interest that specifically inhibited RhaS were expected to decrease lacZ expression from rhaB-lacZ (primary screen), but not from hts-lacZ (secondary screen).

We screened a library of ˜100,000 small-molecule compounds for those that decreased rhaB-lacZ expression in the primary screening strain using a high-throughput β-galactosidase assay. All but three of the 295 plates (384-well) assayed had Z-factors above 0.5, and the remaining three plates had Z-factors above 0.4. The average of the Z-factors for all of the plates was 0.7, indicating that this was an excellent assay. A major contribution to the performance of the assay was that the positive and negative controls had coefficients of variation of only 2-3% over the entire assay.

The inducer for RhaS activation of rhaBAD transcription, L-rhamnose, was added to the assay wells at the same time as the compounds. In this way, rhaB-lacZ expression was uninduced until exposure to the compounds, and it wasn't necessary for pre-formed β-galactosidase to decay before inhibition could be detected. We further studied compounds that resulted in rhaB-lacZ expression levels that were at least three standard deviations below the mean of all of the compounds in the study. This allowed us to focus further efforts on a convenient number of compounds likely to show significant inhibition. These compounds decreased rhaB-lacZ expression levels to between zero and seventy-five percent of the fully induced, non-inhibited control expression levels (data not shown).

The ˜300 most inhibitory compounds from the primary screen (˜0.3% hit rate) were re-assayed with both the RhaS-activated rhaB-lacZ fusion and the LacI-repressed control hts-lacZ fusion (data not shown). IPTG, the inducer of the LacI-repressed hts-lacZ fusion, was added to the assay wells at the same time as the compounds. Similar to addition of the inducer L-rhamnose to the primary screen, this resulted in LacZ expression that was uninduced until exposure to compounds. We ranked each compound by the difference in lacZ expression levels between the RhaS-activated and the LacI-repressed fusions. Expression levels were normalized to the average of the uninduced controls (set to 0%) and the average of the induced controls (set to 100%). Compounds that inhibited expression from the RhaS-activated fusion to a significantly greater extent than the LacI-repressed fusion were likely specific inhibitors of RhaS activity, and were of further interest. We selected the 17 compounds that best fit these criteria for further study as potential specific RhaS inhibitors.

Dose-Dependent Inhibition of RhaS.

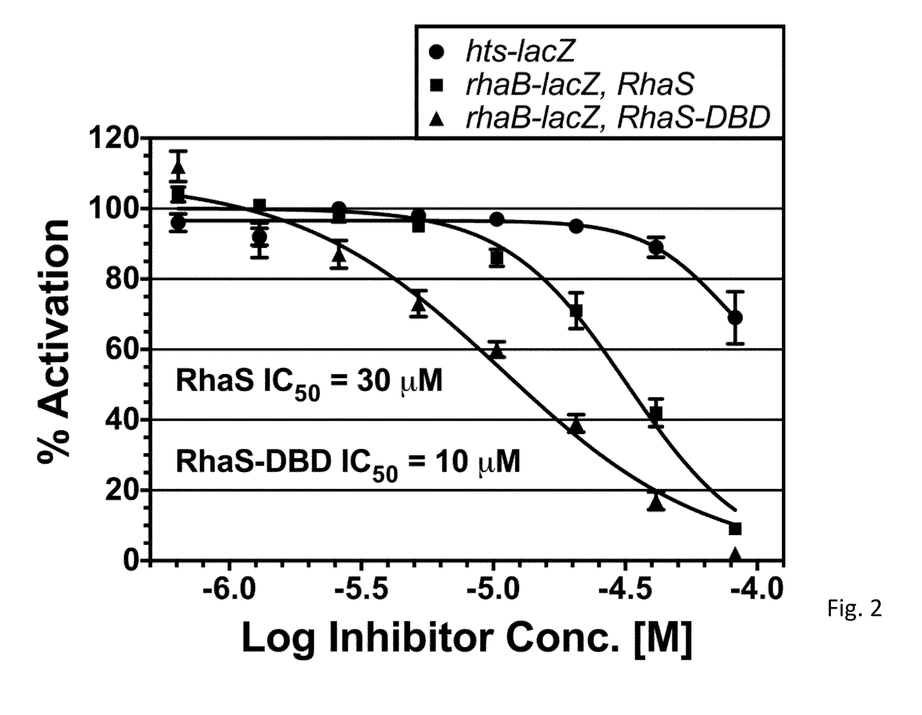

We tested the effects of various concentrations of the 17 potential RhaS inhibitors on activation of the RhaS-activated fusion compared with the LacI-repressed fusion to identify those with dose-dependent inhibition (data not shown). One of the compounds, Compound 2, showed particularly robust, dose-dependent inhibition of the RhaS-activated fusion, and substantially less inhibition of the LacI-repressed fusion. We further describe studies of Compound 1, which is nearly identical in structure to the Compound 2 identified in the screen, was readily available commercially, and inhibited to the same extent as the screen compound (data not shown). We found that Compound 1 inhibited expression of the RhaS-activated fusion to a much greater extent than the LacI-repressed fusion (FIG. 2, circle and square markers). Compound 1 was able to fully inhibit expression of the RhaS-activated fusion, and had an IC₅₀ value of approximately 30 μM. Although there was a small amount of non-specific inhibition, the majority of the Compound 1 inhibition appears to be specific for RhaS. Compound 1 is not structurally related to any of the inhibitors of AraC family activators that have been previously identified.

It was found that Compound 1 inhibited the RhaS DNA-binding domain to the same extent as full-length RhaS. The first step we took toward identifying the mechanism of RhaS inhibition by Compound 1 was to determine whether the RhaS N-terminal domain was required for the inhibitory effect. Proteins are defined as AraC family members if they contain a conserved DNA binding domain that includes two helix-turn-helix motifs, as defined by Prosite entry PS01124 (prosite.expasy.org/PS01124). The RhaS C-terminal DNA-binding domain alone [residues 163 to 278, RhaS(163-278), previously published as RhaS-CTD] is capable of activating transcription of the rhaB-lacZ fusion used in these studies, albeit to approximately a three-fold lower level than full-length RhaS. We therefore compared the effect of Compound 1 on activation of the RhaS-activated rhaB-lacZ fusion by full-length RhaS and RhaS(163-278) (FIG. 2). We found that Compound 1 inhibited RhaS(163-278) to at least the same extent as full-length RhaS. Compound 1 was again able to fully inhibit expression, this time with an IC₅₀ value of approximately 10 μM. This result indicates that the RhaS N-terminal domain, which is required for dimerization and L-rhamnose binding, is not required for Compound 1 inhibition of RhaS. We therefore conclude that Compound 1 specifically inhibits a function of the RhaS DNA-binding domain: likely DNA binding or contacts with the RNA polymerase σ subunit.

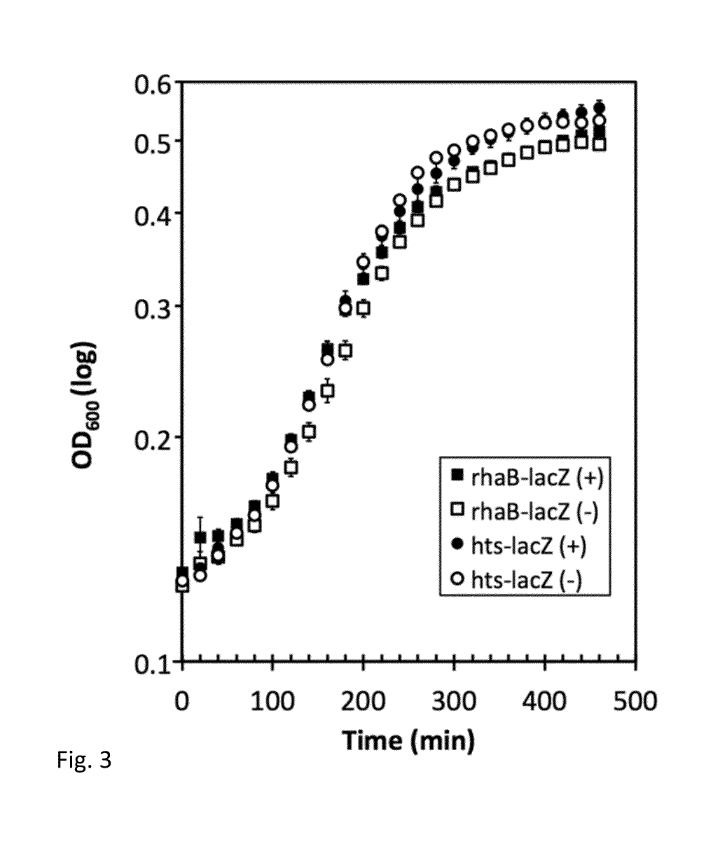

It was found that Compound 1 does not inhibit cell growth. All of our assays to this point were performed in whole cells, thus it was important to test whether Compound 1 had any significant effects on the growth of the bacterial cells. We assayed the growth of the strains carrying the RhaS-activated and the LacI-repressed fusions, grown in the same minimal medium used for the in vivo dose response assays. We found that there was no impact of the inhibitor on the growth of the cells for either strain (FIG. 3). Note that the slight divergence of the plus and minus inhibitor curves at the very end of the growth period is not due to inhibitor toxicity, since the cells grown without inhibitor have the slower growth. This result supports the hypothesis that the inhibition we observed in the in vivo assays was likely due to specific inhibition of RhaS activation.

It was found that Compound 1 inhibits in vitro DNA binding by RhaS and RhaR. In order to perform in vitro DNA binding assays with RhaS, we needed purified protein that was soluble and active. With the exception of low levels of activity from denatured and subsequently refolded protein, we have not previously been able to observe in vitro DNA binding by full-length RhaS, apparently due to its extremely low solubility. Two modifications were required to obtain soluble and active RhaS for these studies. First, we used a RhaS variant, RhaS L201R, which binds DNA more strongly than wild-type RhaS. The second modification was to fuse a GB1^{basic} solubility-enhancement tag to the C-terminus of full-length RhaS L201R, yielding RhaS L201R-GB1^{basic} (referred to as RhaS-GB1^{201b} for brevity). The “basic” variant of GB1 was necessary to prevent tight binding between RhaS and GB1 that blocked DNA binding (Skredenske, Deng and Egan, unpublished). RhaS-GB1^{210b} is soluble and binds DNA in vitro, with increased DNA binding in the presence of L-rhamnose (Deng and Egan, unpublished), as expected for functional RhaS based on previous studies.

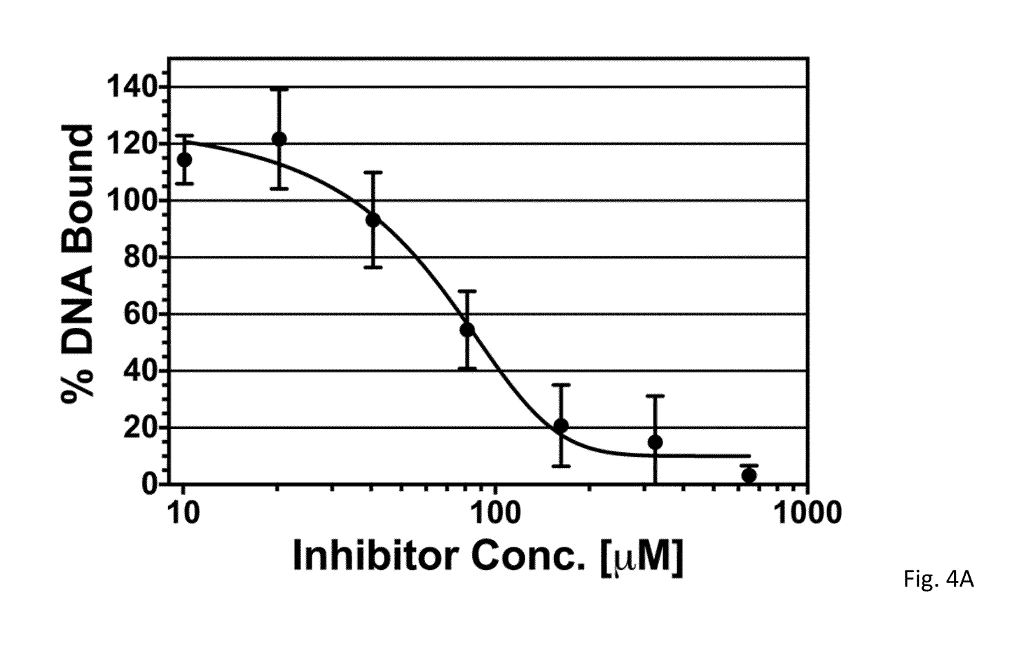

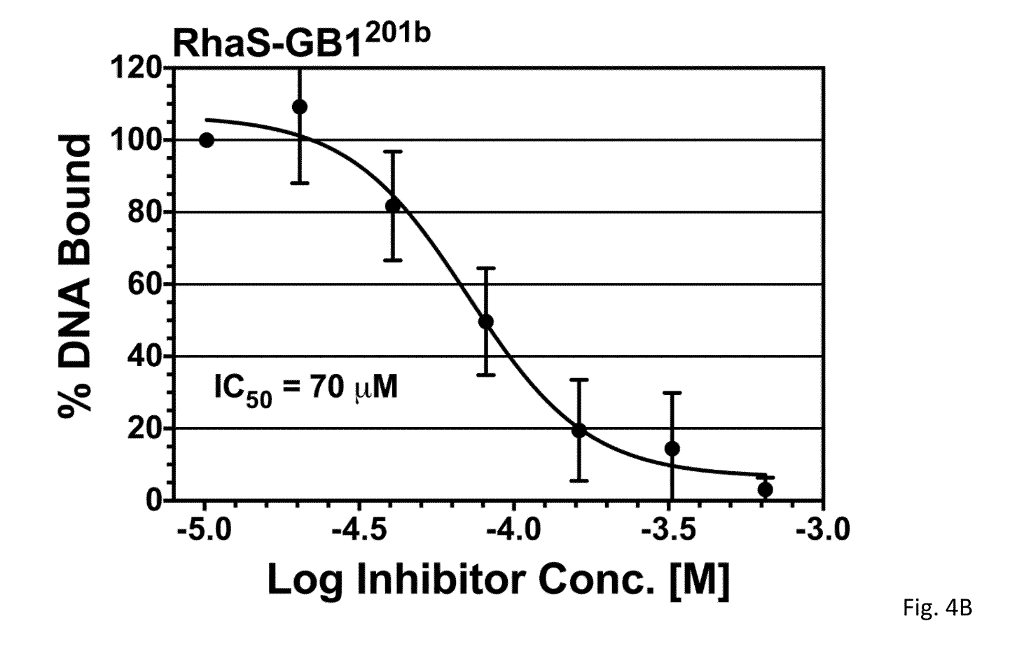

We used the electrophoretic mobility shift assay [EMSA] to investigate whether Compound 1 inhibited in vitro DNA binding by RhaS-GB1^{201b}. We incubated RhaS-GB1^{201b} with dsDNA containing the RhaS binding site sequence from the rhaBAD promoter region (this includes binding sites for both monomers of the RhaS-GB1^{201b} dimer) in the absence or presence of various concentrations of OSSL_{—}051168. We found that Compound 1 was able to fully inhibit DNA binding by RhaS-GB1^{201b} in a dose-dependent manner (FIG. 4). We calculated an IC₅₀ of approximately 70 μM, which is in reasonable agreement with the 30 μM IC₅₀ calculated from the in vivo assays of RhaS inhibition, especially considering the many experimental differences. From this result, we conclude that the inhibition of RhaS activity observed in in vivo assays was likely due to Compound 1 blocking the ability of RhaS to bind to DNA.

We also tested the ability of Compound 1 to inhibit DNA binding by the RhaR protein. As mentioned above, although RhaS and RhaR both activate transcription in response to the effector L-rhamnose, they are only 30% identical to each other. The identity in their DNA binding domains is approximately the same, at 34% amino acid identity. E. coli AraC family activators have pairwise amino acid identities that range from single digits to 56%, thus RhaS and RhaR share an intermediate level of identity

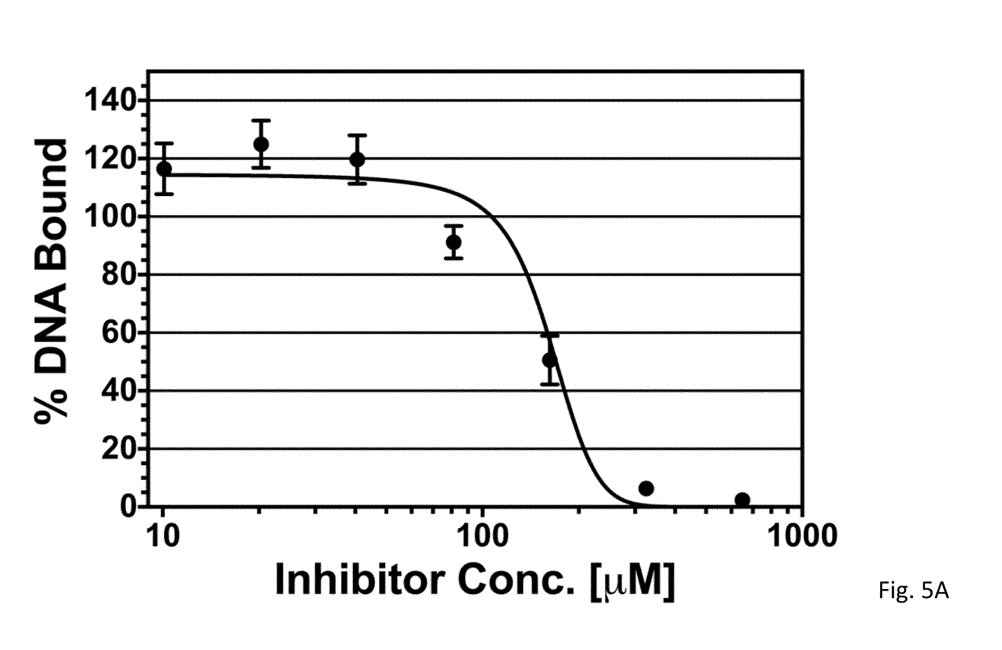

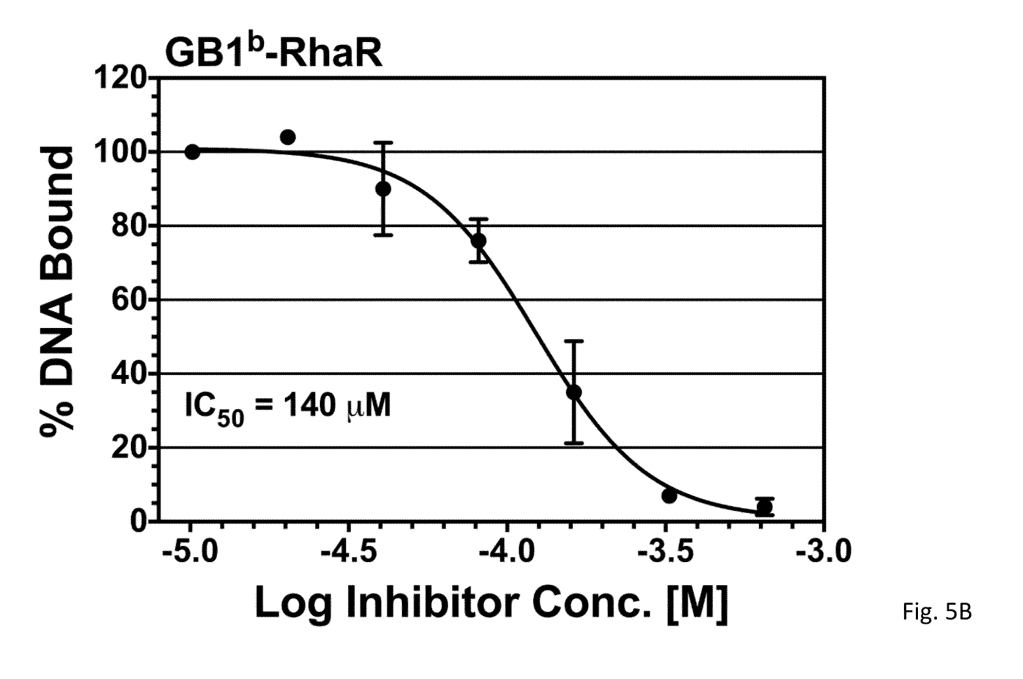

We previously purified RhaR as a tripartite fusion protein with an intein domain and a chitin-binding domain, however the yield of purified protein was extremely low. Here we used a RhaR fusion protein with GB1^{basic}, GB1^{b}-RhaR, which gave a much higher protein yield, and was active for DNA binding—showing the expected increase in DNA binding in the presence of L-rhamnose (Deng and Egan, unpublished). We found that Compound 1 inhibited DNA binding by purified GB1^{b}-RhaR protein to approximately the same extent as RhaS-GB1^{201b} protein (FIG. 5). Compound 1 was able to fully inhibit GB1^{b}-RhaR binding to DNA, with an IC₅₀ value of approximately 140 μM. The concentration of OSSL_{—}051168 required for 50% inhibition of GB1^{b}-RhaR was approximately two-fold higher than that required for inhibition of RhaS-GB1^{201b}, indicating that Compound 1 has slightly higher specificity for RhaS.

It was found that Compound 1 does not inhibit DNA binding by non-AraC family proteins. Our results indicated that Compound 1 inhibits DNA binding by both the RhaS and RhaR proteins. We used two unrelated proteins, the cyclic AMP receptor protein (CRP) and the Lac repressor protein (Lad), to test whether Compound 1 was specific for inhibition of RhaS and RhaR (and perhaps other AraC family proteins), or was broadly inhibitory toward DNA binding proteins. CRP and Lad each share only 10-12% sequence identity with RhaS and RhaR. They both bind to DNA using helix-turn-helix motifs, but neither contains the two helix-turn-helix motifs (per monomer) that is characteristic of AraC family proteins. Broad inhibition of DNA binding by Compound 1 would be expected to have a major impact on cell growth; therefore, the finding that Compound 1 had minimal impact on growth of the strains used for our in vivo studies (FIG. 3) was the first evidence that broad inhibition was unlikely.

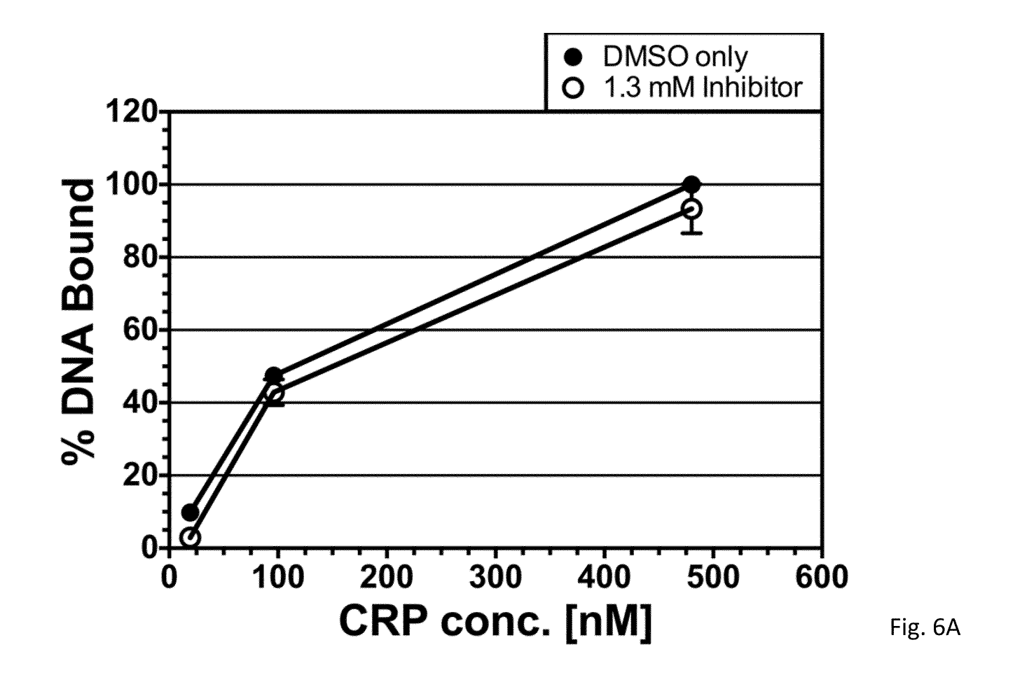

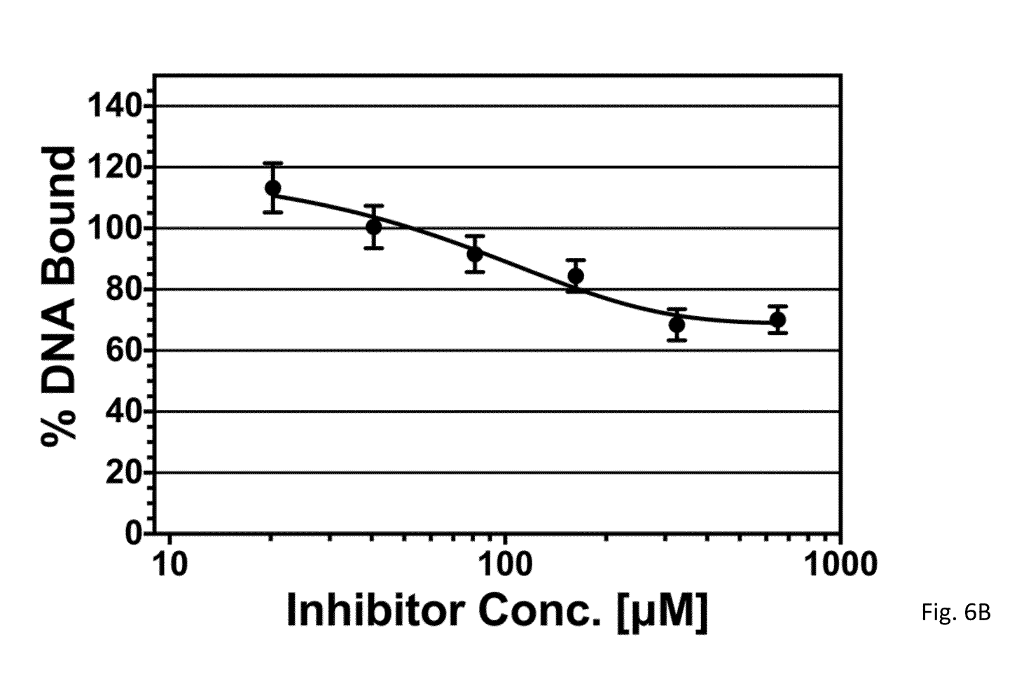

We first tested the effect of Compound 1 on DNA binding by CRP. EMSA reactions were performed with varying concentrations of purified CRP protein. The Compound 1 concentration (1.3 mM) was two-times higher than the concentration required in in vitro assays to nearly eliminate RhaS binding to DNA. We found that even at this very high concentration of Compound 1, approximately 94% of the CRP DNA binding was retained (FIG. 6A). Thus, Compound 1 resulted in only a very slight inhibitory effect on DNA binding by CRP.

As a second test of whether Compound 1 inhibition is specific for AraC family activators, we tested the lac repressor protein, LacI (FIG. 6B). EMSA assays were again performed; in this case various dilutions of Compound 1 (starting at 650 μM) were added to reactions with Lad protein at a concentration just sufficient to shift nearly the entire DNA band. The highest concentrations of Compound 1 had only a slight effect on Lad DNA binding, with approximately 70% of DNA binding retained. Note that this in vitro test of Lad binding to DNA was not redundant with the in vivo hts-lacZ reporter assay, since Lad was in its induced state in the reporter assay, and therefore not bound to DNA.

Taken together, the CRP and Lad results indicate that Compound 1 doesn't simply inhibit the activity of all DNA binding proteins, and further that not all helix-turn-helix containing proteins are inhibited. It has yet to be determined whether Compound 1 is specific for only RhaS and RhaR. However, the above finding that Compound 1 inhibits RhaR, a protein with only 30% identity with RhaS, suggests the possibility that Compound 1 might inhibit DNA binding by additional AraC family proteins, perhaps including activators of virulence factor expression in bacterial pathogens.

The screen identified inhibitors (Compound 1 and Compound 2) of AraC family activators. Members of the AraC family of transcriptional activators have excellent potential as targets for novel antibacterial agents based on the fact that they are required for the expression of virulence factors in many bacterial pathogens. In addition, many of the bacteria that require AraC family activators to cause human disease pose serious health threats due to their resistance to currently available antibiotics, including Enterococci, Mycobacterium tuberculosis, and Pseudomonas aeruginosa.

We developed and validated a novel high throughput assay to screen for small molecule inhibitors of the E. coli AraC family activator protein RhaS. The in vivo assay achieved specificity for RhaS inhibitors through comparison of expression levels from two reporter constructs; one that was transcriptionally activated by RhaS, and a second that was transcriptionally repressed by the non-AraC family repressor Lad. The LacI-repressed reporter enabled us to identify and eliminate non-specific inhibitors from consideration. Of the compounds identified in our screen, we further investigated Compound 1, which was an especially effective inhibitor of RhaS. Compound 1 is not structurally related to any of the previously identified small molecule inhibitors of AraC family activators, and thus is a novel inhibitor.

Compound 1 has specificity and potency. Given that Compound 1 was identified through a whole-cell high throughput screen, and has not undergone chemical optimization, its potency is reasonable. The following results indicate that the majority of the Compound 1 inhibition that we observed in whole cell assays was specific for AraC family proteins. First, we found very little inhibition of expression from the control fusion, hts-lacZ, indicating that there wasn't significant inhibition of β-galactosidase enzyme activity or of any processes that impacted transcription or growth rate. The absence of an impact of Compound 1 on E. coli growth rate was more directly confirmed by comparing cell growth rates in the absence and presence of Compound 1. Finally, we determined that Compound 1 had only very little effect on DNA binding by two proteins unrelated to the AraC family and unrelated to each other, CRP and Lad. This result suggests that Compound 1 does not inhibit by a non-specific mechanism such as binding to DNA to block protein binding. Since both CRP and Lad use helix-turn-helix motifs to contact DNA, this finding also suggests that Compound 1 doesn't generally block DNA binding by helix-turn-helix containing proteins. Therefore, it appears that inhibition by Compound 1 involves binding to some feature that is unique to RhaS, RhaR, and possibly additional AraC family activator proteins.

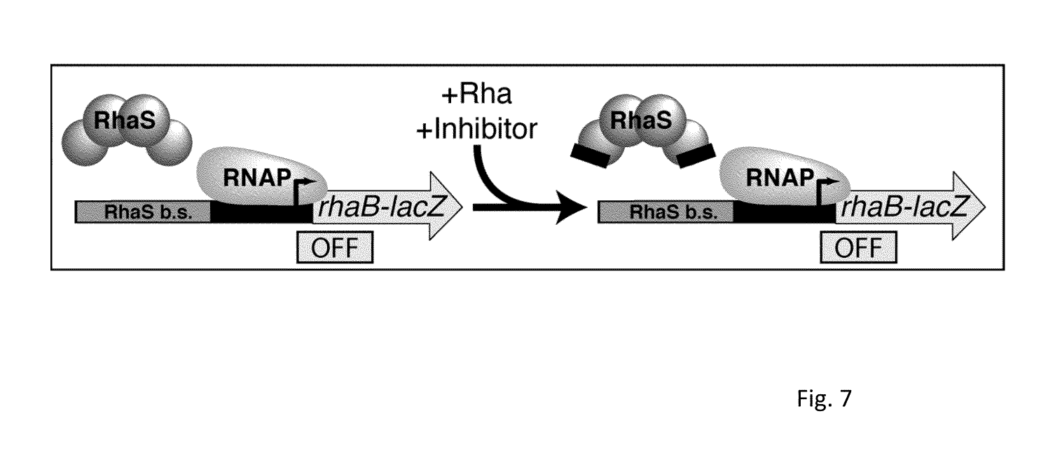

Compound 1 inhibits the conserved AraC family DNA binding domain. In addition to their conserved DNA binding domains, the majority of AraC family proteins contain a second, non-conserved, domain that regulates the activity of the DNA binding domain, and in some cases imparts dimerization. Compound 1 inhibits the activity of the more conserved of the two RhaS domains, the DNA binding domain. Thus, Compound 1 might inhibit the activity of additional AraC family proteins; and indeed, we found that Compound 1 also inhibited DNA binding by RhaR. Taken together, our results lead to the hypothesis that the Compound 1 mechanism of action involves binding to the DNA binding domain of AraC family proteins and blocking their ability to bind to DNA (FIG. 7).

### Experimental 2

Bacterial Strains, Plasmids and Growth Conditions.

Escherichia coli strains were grown in Tryptone-Yeast extract (TY) broth (0.8% Difco tryptone, 0.5% Difco yeast extract, and 0.5% NaCl, pH 7.0). Cultures for phage Plvir infection (generalized transduction) were grown in TY broth supplemented with 5 mM CaCl₂. Difco Nutrient Agar (1.5% agar) (Becton, Dickinson and Company, BD, Cockeysville, Md.) was used routinely to grow E. coli strains on solid medium. S. flexneri was cultured in Tryptic soy broth (TSB, pH 7.0) (BD), Luria-Bertani broth (1% tryptone, 0.5% yeast extract and 1% NaCl) or on Tryptic soy agar (TSA, 1.5% agar) (BD) containing Congo red dye (0.025%). All Shigella strains were picked as red colonies from TSA plates containing Congo red. Cultures were grown at 37° C. unless otherwise specified. Appropriate antibiotics (ampicillin, 100 ng/ml; tetracycline, 20 μg/ml; chloramphenicol, 30 μg/ml; and gentamycin, 10 μg/ml) were used as indicated. The entire open reading frame of all cloned genes was sequenced on both strands. L-929 mouse fibroblast cells (ATCC, Manassas, Va.) were routinely cultured in RPMI 1640 tissue culture medium (Mediatech, Manassas, Va.) supplemented with 10 μg/ml gentamycin (MP Biomedicals, Santa Ana, Calif.) and 5% fetal bovine serum (Thermo Fisher, Waltham, Mass.) in a humidified 5% CO₂ incubator at 37° C.

Strain Construction for In Vivo Dose-Response Studies.

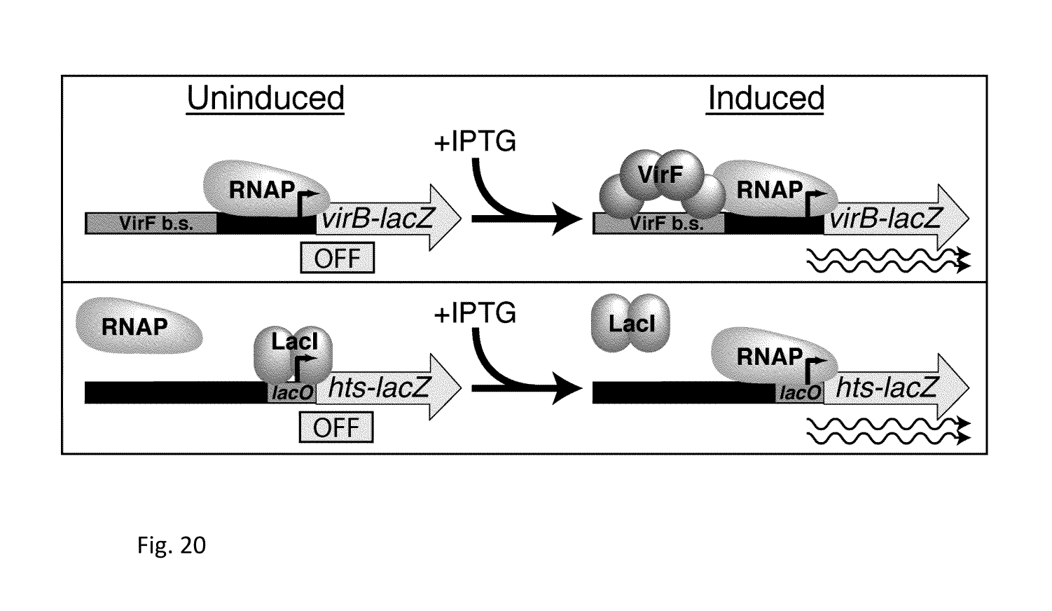

For dose-response studies, a virB-lacZ reporter strain was constructed that allowed IPTG (isopropyl-β-D-thiogalactopyranoside) induced expression of VirF from plasmid pHG165. To construct this virB-lacZ reporter strain, kMAD102 was isolated from strain AB97, used to infect strain ECL116, and single copy lysogens were identified. Plvir-mediated generalized transductions were then used to add malP::lacI^{q} and recA::cat to this strain, as described previously. The virF gene was amplified (Forward oligo, Reverse oligo), digested with EcoRI and PstI and then ligated into the same sites of plasmid pHG165. The resulting plasmid, pHG165virF was transformed into SME4259 to make SME4382 (FIG. 20, Top). A control strain, SME3359, which carries a single copy hts-lacZ reporter fusion in the chromosome and a plasmid pHG165lacI that expresses LacI protein (FIG. 20, Bottom), was constructed. Briefly, the hts-lacZ reporter fusion consists of a synthetic promoter that is repressed by Lad. The control strain is isogenic with SME4382, except that it does not carry malP:lacI^{q} and therefore Lad is expressed from pHG165lacI regardless of the presence of IPTG.

In Vivo Dose-Response Experiments in E. coli.

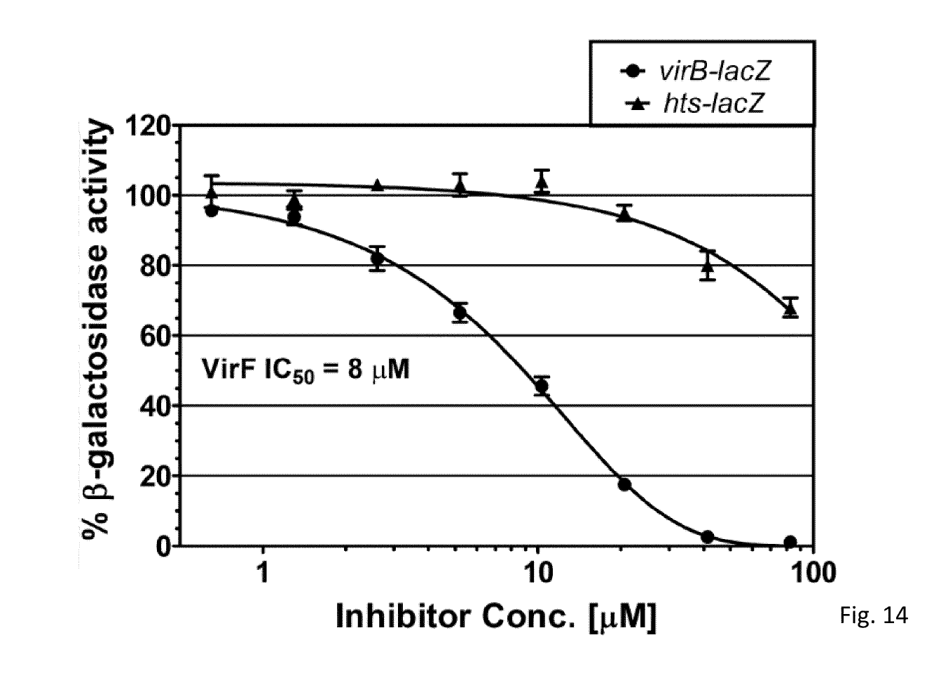

In vivo dose-response assays were performed using a procedure described previously. In brief, cultures of SME4382 and SME3359 grown overnight were diluted to an OD₆₀₀ of ˜0.1. SE-1 (1-butyl-4-nitromethyl-3-quinolin-2-yl-4H-quinoline, formerly OSSL_{—}051168, referred to as Compound 1) was obtained from eMolecules, Inc. (Solana Beach, Calif.; catalog #3761-0013) or Princeton BioMolecular Research (Princeton, N.J.; catalog #OSSL_{—}051168) and was dissolved in 100% DMSO. Bacterial cultures were mixed with varying concentrations of SE-1 (2.2% DMSO, final), induced with 6.5 mM IPTG for 3 h at 37° C., lysed and β-galactosidase activity was measured. Uninduced (no IPTG and no SE-1) and uninhibited (6.5 mM IPTG and no SE-1) controls were included for each strain and used to normalize β-galactosidase activity, as described previously. IC₅₀ values were calculated and graphs were drawn using Prism (GraphPad, La Jolla, Calif.). FIG. 14 shows the data, where error bars represent the standard error of the mean of three independent experiments with two replicates each.

E. coli Growth Curves.

To test the impact of SE-1 on growth of the bacterial strains used for in vivo dose-response assays (SME4382 and SME3359), we compared growth rates in the presence and absence of SE-1, without addition of rhamnose. Briefly, the cells were grown at 37° C. with shaking in MOPS-buffered minimal medium with glycerol and plus 6.5 mM IPTG, in 24-well plates, with or without: SE-1 (44 uM SE-1, 0.3% DMSO), or DMSO (0.3%) in a PowerWave XS plate reader equipped with KC⁴ data analysis software (BioTek Instruments).

Heterologous Expression and Purification of VirF.

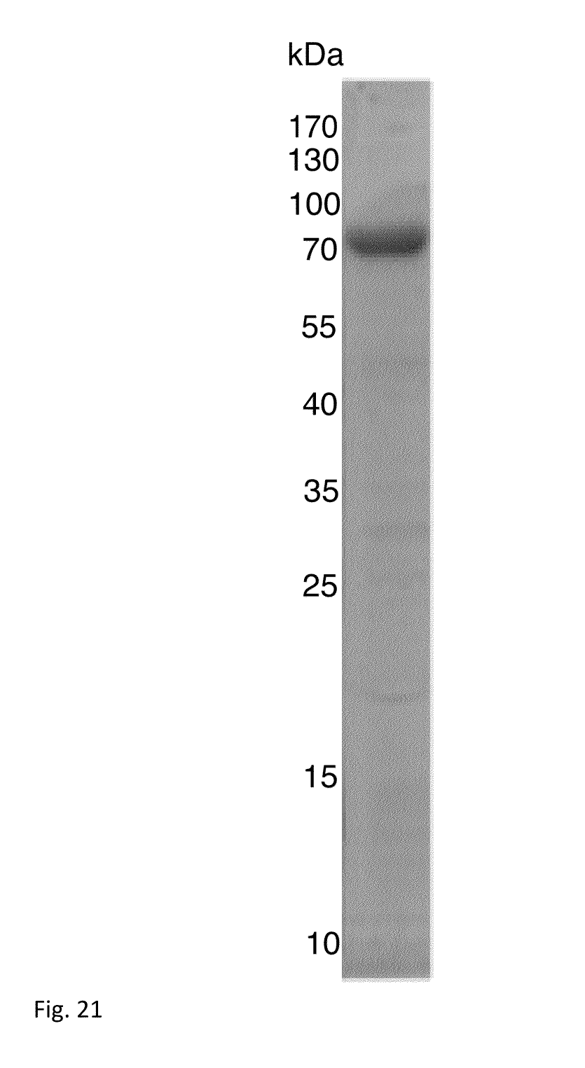

The IPTG inducible MBP-VirF expression plasmid, pMALvirF, was constructed by cloning virF downstream and in-frame with malE (encoding maltose binding protein, MBP) in the vector pMAL-c2X (New England Biolabs, Beverly, Mass.). MBP-VirF protein (referred to as VirF for simplicity) was purified using amylose affinity chromatography at 4° C. Briefly, pMALvirF was transformed into E. coli strain KS 1000 (New England Biolabs). The cells were grown to an OD₆₀₀ of 0.5, transferred to 15° C., 0.1 mM IPTG was added, and then incubated overnight with shaking Cells were harvested and resuspended in 10 ml of binding buffer (20 mM Tris, 500 mM NaCl, 1 mM EDTA 1 mM DTT, pH 7.4). Cells were lysed by three freeze thaw cycles with lysozyme (0.4 mg/ml), tris (2-carboxyethyl)phosphine (TCEP, 1 mM) and phenylmethylsulfonyl fluoride (PMSF, 1 mM), at −80° C., followed by sonication and then centrifuged to remove cell debris. The supernatant was applied with a BioLogic LP chromatography system (Bio-Rad Laboratories) to an amylose resin column (New England Biolabs), pre-equilibrated with 80 ml binding buffer, the column was washed with 120 ml binding buffer. VirF was eluted with 40 ml of elution buffer [binding buffer plus 15% glycerol (w/v) and 10 mM maltose], and used directly for subsequent assays. The purified protein was greater than 90% pure (FIG. 21), although some older preps showed partial breakdown to MBP and non-fusion VirF. EMSAs did not show any evidence of non-fusion VirF binding to DNA (data not shown), suggesting that the non-fusion VirF protein was inactive (likely due to aggregation).

Electrophoretic Mobility Shift Assays.

Binding of purified VirF to a DNA fragment containing the VirF binding site at virB was analyzed by electrophoretic mobility shift assay (EMSA) (68), in the absence or presence of SE-1. DNA probes were generated by hybridizing (69) the following oligonucleotides (oligos, Eurofins MWG Operon, Huntsville, Ala.): A short IR700-labeled LUEGO oligo, a top oligo containing the virB binding site, and a bottom oligo that was complementary to both of the preceding oligos. Reactions and electrophoresis were performed without any of the described additives, however all reactions contained 10% DMSO (used to dilute SE-1). Gels were scanned using an Odyssey infrared imager (LI-COR, Lincoln, Nebr.), and quantified using Odyssey software, version 3.0.30. Graphs were drawn using Prism (GraphPad).

Fluorescence-Based Thermal Shift Assay.

Binding of SE-1 to purified VirF was tested by comparing the VirF melting temperature (Tₘ) in the absence and presence of SE-1 using a thermal shift assay with the fluorescent dye Sypro Orange (Molecular Probes). This method is also known as differential scanning fluorimetry or DSF. As a control, we tested binding of SE-1 to the purified, unrelated, MBP-NS1-NTD protein (greater than 95% pure). Reactions contained (final concentrations): 1 μM protein; 20×Sypro Orange (a 250-fold dilution of the purchased 5,000× stock solution); 0.76×EMSA buffer (7.6 mM Tris-acetate, pH 7.4, 0.76 mM KEDTA, 38 mM KCl, and 0.76 mM DTT); 4% DMSO; and 0 or 80 μM SE-1. The reactions were carried out in 0.1 ml PCR low profile 8-tube strips with optically clear flat caps (USA Scientific) in a StepOnePlus™ Real-Time PCR System (Applied Biosystems), heating from 25 to 99° C. in increments of 0.8° C. The data was analyzed using the Boltzmann sigmoidal model in the software Prism (GraphPad). Data points before and after the fluorescence intensity minimum and maximum, respectively, were excluded from fitting. The ΔTₘ values are the average of three independent experiments with two replicates in each experiment. A single, representative, experiment is shown.

Shigella virB-lacZ Reporter Gene Assays.

Colonies of S. flexneri strain BS536 that were red on TSA plates containing Congo red were grown overnight in Luria-Bertani broth at 30° C. to maintain low basal expression from the virB promoter. The overnight grown cultures were diluted 1:100 into 10 ml of the same medium with either 0.3% DMSO or different concentrations of SE-1 dissolved in DMSO (final concentration 0.3% DMSO). Cultures were grown at 37° C. in a shaking incubator to an OD₆₀₀ of ˜0.4, centrifuged to pellet the cells and then resuspended in Z buffer for a two-fold concentration of the cells. Control cultures were grown at 30° C., a temperature at which virF expression is not induced (37), to illustrate the basal level of expression from the virB promoter region. β-Galactosidase assays were performed, with incubation with the substrate o-nitrophenyl-β-D-galactopyranoside (ONPG) was at room temperature. Activities are presented as a percentage of the uninhibited, DMSO-only control sample. Three independent assays were performed with two replicates in each assay.

Shigella Gene Expression Analysis.

Real-time quantitative reverse transcription PCR (qRT-PCR) was performed to test the effects of SE-1 on the expression of the VirF regulated genes virB, icsA, icsB and ipaB. The gapA and rrsA transcripts were used as internal controls. These are constitutively expressed Shigella genes commonly used to normalize mRNA levels. S. flexneri strain SME4331, which carries a null mutation in ipgD (ipgD), was grown at 30° C. overnight in TSB with ampicillin, diluted into the same medium to an OD₆₀₀ of ˜0.1, and 1 ml aliquots further grown at 30° C. (DMSO only) or at 37° C. (SE-1 at 20 or 40 μM, or DMSO only, all 0.3% DMSO). Cells were grown to an OD₆₀₀ of 1.0 (˜2.5 h) and RNA was isolated using an RNeasy MiniElute Cleanup Kit (Qiagen, Germantown, Md.), with DNA contamination eliminated using a Turbo DNase Kit (Ambion, Austin, Tex.). Complementary DNA (cDNA) was synthesized by random priming using a cDNA Reverse Transcription Kit (Applied Biosystems, Foster City, Calif.). Primers were tested for PCR specificity using genomic DNA template to ensure a single amplicon of the expected size for each primer pair, and validated using 5-fold dilutions of cDNA (2000 to 0.0256 ng) to ensure a linear plot of cycle threshold (Ct) versus cDNA concentration. Validated primer sets were used to test mRNA profiles in reactions that contained 10 ng of cDNA, 10 μl of SYBR green master mix (Applied Biosystems), specific primers (0.5 μM each) and water to a final volume of 20 μl, using a Step One Plus Real-Time PCR System (Applied Biosystems). The data was analyzed using the 2^{-ΔΔCt} method, and also by Applied Biosystems real-time analysis software. The data analyzed by the 2^{-ΔΔCt} method is reported, however, both analyses produced comparable results. In brief, ΔΔCt values [ΔΔCt=(Ct,_{Target}−Ct,_{Internal Control})_{SE-1}−(Ct,_{Target}−Ct,_{Internal Control})_{no SE-1}] were separately obtained for each sample relative to two internal controls, gapA and rrsA. The ΔΔCt values relative to the two internal controls for each sample were averaged and then used to calculate the relative expression levels (2^{-ΔΔCt}). As a control, we also normalized each internal control to the other internal control. Example data and calculated values are shown in Table S1. Control reactions used templates generated without reverse transcriptase to ensure low genomic DNA contamination. To ensure that products were not likely to be due to primer dimer artifacts, additional control samples were performed in which reaction mixtures contained primers alone (no template).

Shigella Growth Curves.

The S. flexneri ipgD⁻ strain (SME4331) was grown overnight at 30° C. and then diluted to an OD₆₀₀ of ˜0.1 in TSB supplemented with ampicillin. SE-1 was diluted in DMSO and added to the SME4331 cultures at final concentrations of 0, 20, or 40 μM SE-1 and 0.3% DMSO. These cultures were grown at 37° C. in 24-well plates containing 1.2 ml of each sample in the temperature-controlled chamber of a PowerWave XS plate reader equipped with KC⁴ data analysis software (BioTek Instruments). Bacterial growth was continued at 37° C. for 8 h with shaking and OD₆₀₀ was monitored every 5 min for growth curves (20 min time intervals were plotted).

Shigella Invasion Assays.

S. flexneri ipgD⁻ (SME4331) and mxiH⁻ (SB116) mutant strains were grown overnight at 30° C. and then diluted to an OD₆₀₀ of ˜0.1 into TSB plus ampicillin. SE-1 (in DMSO) was added to the cultures at final concentrations of 0, 20, or 40 μM SE-1 and 0.3% DMSO. These cultures and SB116 (no SE-1) were grown at 37° C. in 24-well plates (each with 1.2 ml), in the temperature-controlled chamber of a PowerWave XS plate reader (BioTek Instruments). Bacterial growth was continued at 37° C. with shaking and OD₆₀₀ monitored every 15 min. A separate sample of SME4331 was grown at 30° C. L-929 cells (mouse fibroblasts, used previously for Shigella invasion assays) that had been grown overnight to ˜90% confluency in 24-well plates were washed 3 times with 1 ml of RPMI each (without FBS or antibiotics), and the final wash was removed by aspiration. (All L-929 incubation steps were carried out at 37° C. in a 5% CO₂ humidified atmosphere.) When the OD₆₀₀ of the bacterial cultures reached 1.0 (˜2.5 h), samples were diluted 100-fold in pre-warmed RPMI (without FBS or antibiotics) and then 300 μl was added to each monolayer of L-929 cells, for a multiplicity of infection of roughly 20. In a control sample, 40 μM SE-1 and bacteria grown at 37° C. without SE-1 were added at the same time to L-929 cells. The 24-well plate was centrifuged (2000×g) for 5 min at room temperature to initiate bacterial-host cell contact, incubated for 45 min at 37° C., washed three times with 1 ml RPMI containing 50 μg/ml gentamycin (or without gentamycin for the control), and incubated at 37° C. for 2 h. Cells were again washed three times with 1 ml RPMI and then lysed in 100 μl of PBS (Mediatech) containing 0.1% triton X-100 (Promega, Madison, Wis.) for 10 min at room temperature. The 100 μl lysates from the gentamycin treated and untreated L-929 cells were mixed with 400 μl TSB, and 100 μl of 1:50 and 1:250 further dilutions were plated onto TSB agar containing ampicillin. Plates were incubated overnight at 37° C. and colonies were counted. The number of colonies obtained from gentamycin treated samples was divided by the number of colonies from control samples without gentamycin and multiplied by 100 to calculate the invasion index.

AlamarBlue® assays were performed as a control to test whether SE-1 had any impact on the metabolic activity of the host cells, as follows: L-929 cells were seeded into 96-well plates and grown overnight to ˜90% confluency. SE-1 was serially diluted in RPMI supplemented with 10 μg/ml gentamycin and 5% FBS, added to the wells, and incubated at 37° C. for 3 h. Cells were washed with 200 μl Hank's buffered salt solution (Mediatech) and incubated with 200 μl RPMI (without phenol red) containing 10% AlamarBlue® (also known as Resazurin, Invitrogen, Grand Island, N.Y.). Following 8 h incubation at 37° C., the absorbance was measured at 570 nm and 600 nm using a PowerWave XS plate reader, and the metabolic activity of the cells (as a percentage of untreated controls) was calculated using the following formula provided by the manufacturer: (117,216×A₅₇₀−80,586×A₆₀₀)/(117,216×A′₅₇₀−80,586×A′₆₀₀)×100, where A is the absorbance of test wells, and A′ is the absorbance of positive control wells (mock-infection).

SE-1 Inhibited VirF Activation of a virB-lacZ Fusion in E. coli.

The small molecule SE-1 (FIG. 1A) was previously found to inhibit DNA binding and transcription activation by the E. coli AraC family proteins RhaS and RhaR. Given that our findings indicated that SE-1 interacted with the relatively conserved DNA binding domain of these AraC family proteins, we tested whether it might also inhibit VirF, an AraC family activator required for virulence in Shigella. SE-1 inhibition of transcription activation by VirF was first tested using cell-based assays in E. coli. We performed β-galactosidase assays in the absence or presence of inhibitor in a non-pathogenic strain of E. coli that carried a single copy of the lacZ reporter gene under the control of the virB promoter, virB-lacZ, in the chromosome (FIG. 20, Top) and a plasmid with IPTG-inducible expression of VirF. A control strain carried plasmid-encoded Lad and the lacZ reporter gene under the control of the synthetic, LacI-repressible hts promoter, hts-lacZ (FIG. 20, Bottom). This control strain served to distinguish inhibition of overall transcription or β-galactosidase activity, for example, from selective inhibition of transcription activation by VirF. IPTG was added to the cells (to induce VirF expression or release Lad from repressing hts-lacZ) at the same time as inhibitor. Thus, β-galactosidase was maintained at an uninduced level until inhibitor was added, and it was not necessary to wait for decay of preformed β-galactosidase to detect inhibition. Expression of lacZ from uninduced and induced controls was set to 0% and 100%, respectively, thus normalizing the effect of inhibitor on the VirF-activated fusion and the LacI-repressed fusion.

SE-1 inhibition of transcription activation by VirF was predicted to decrease lacZ expression from virB-lacZ with little or no effect on hts-lacZ expression. In contrast, a decrease in lacZ expression from both virB-lacZ and hts-lacZ would indicate that inhibition was not specific for VirF. We found that SE-1 showed a substantially greater dose-dependent inhibition of the VirF-activated lacZ fusion than the LacI-repressed fusion (FIG. 14) Inhibition of the expression from the VirF-activated fusion had an IC₅₀ value of 8 04. This is a typical potency for a hit from a high throughput screen (low μM to high nM potency range) that has not yet been chemically optimized and more potent than the IC₅₀ value of 30 μM found in similar assays with RhaS. Similar to the previous report in which we used hts-lacZ as a control, there was also some non-specific inhibition of the LacZ expression from the LacI-repressed fusion at higher doses of SE-1. Overall, these cell-based assays suggest that SE-1 inhibits transcription activation by VirF, at least in E. coli, with reasonable selectivity.

SE-1 Did not Impact E. coli Growth.

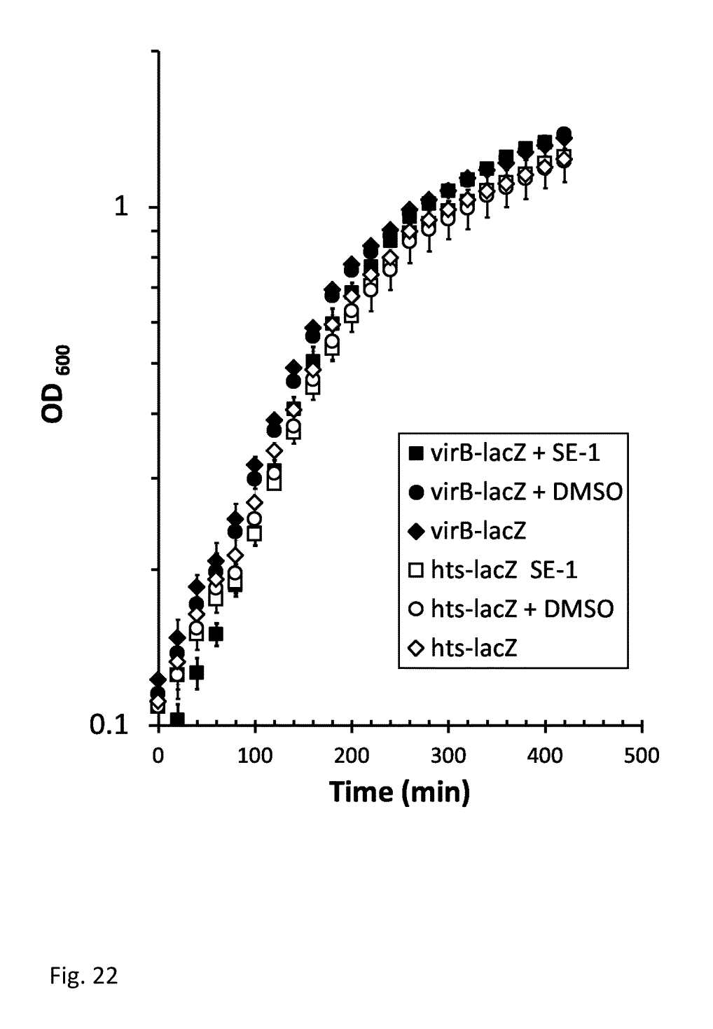

Bacterial growth assays were performed to ensure that the observed inhibition of VirF activation could not be attributed to toxicity toward the strains of E. coli used in the cell-based reporter assays. Strains carrying the VirF-activated and LacI-repressed fusions were grown for 8 h at 37° C. in the same minimal medium used for the in vivo dose-response assays. We found no detectable effect of SE-1 on growth of either strain, indicating that the inhibition observed in the whole cell assays could not be attributed to effects on cell growth (FIG. 22). As a control, we tested whether 0.3% DMSO (the solvent for SE-1) had any impact on the growth rate of these strains of E. coli, and found that it did not (FIG. 22). The finding that the tested concentrations of SE-1 had no detectable effects on the bacterial growth rates further supports the conclusion that SE-1 exhibits selectivity for VirF inhibition.

SE-1 Inhibited In Vitro DNA Binding by VirF.

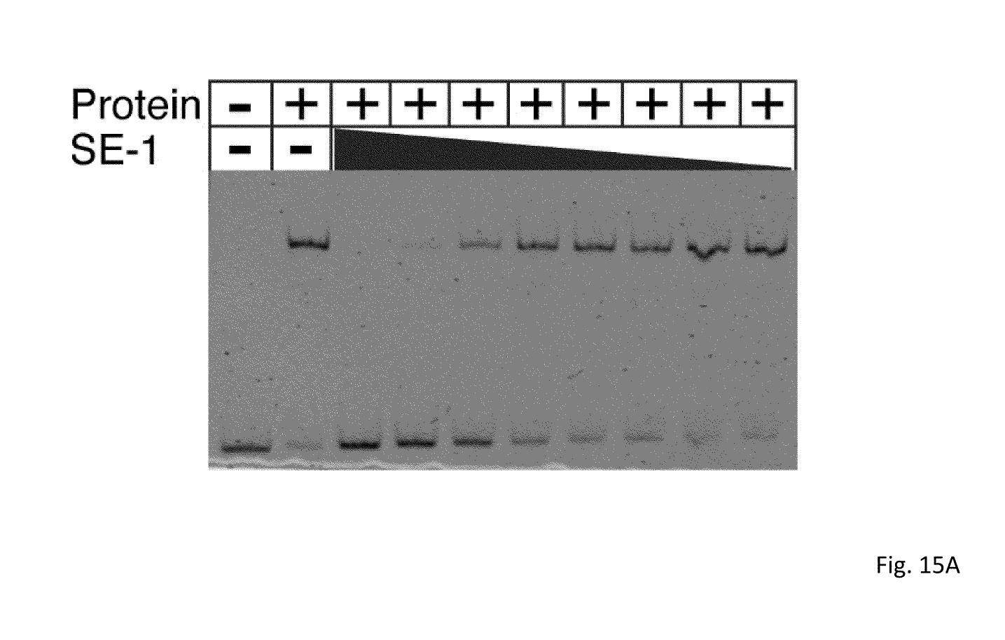

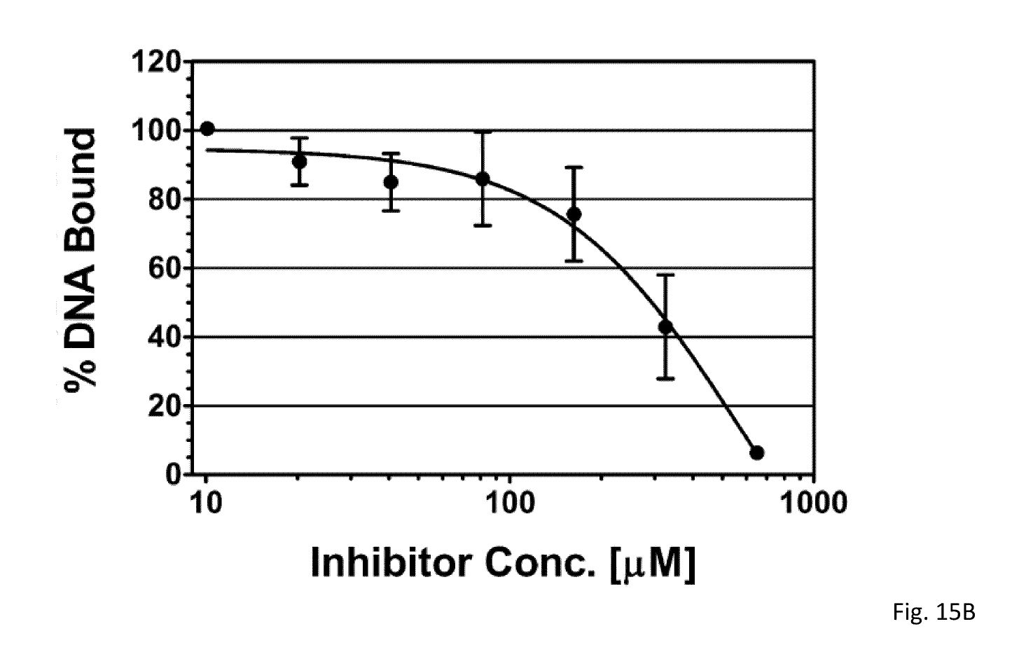

Electrophoretic mobility shift assays (EMSAs) were performed to investigate whether, similar to RhaS and RhaR, SE-1 inhibits DNA binding by purified VirF protein (MBP-VirF). The VirF protein was purified using amylose affinity chromatography, and the DNA tested included the VirF binding site sequence from the virB promoter region. Our results indicate that SE-1 was able to fully inhibit DNA binding by VirF in a dose-dependent manner (FIGS. 15A-15B). The concentration of SE-1 required for half-maximal inhibition of DNA binding was higher than the IC₅₀ for the cell-based assays. However, it was not possible to calculate an accurate IC₅₀ value from the DNA binding assays due to limitations of the detection method sensitivity, solubility of SE-1, and residual aggregation of the purified VirF protein. We previously showed that SE-1 did not inhibit DNA binding by the Lad or CRP proteins. Lad and CRP are not members of the AraC family, and each are founding members of their own protein families. Overall, our EMSA results indicate that SE-1 can block the ability of VirF to bind to its specific DNA site.

SE-1 Binds Directly to VirF.

The finding that SE-1 blocked DNA binding by VirF but not by the non-AraC family proteins Lad or CRP suggested that SE-1 might bind directly to VirF. (At least in the simplest case, SE-1 binding to DNA would be expected to inhibit DNA binding by any protein tested.) To test the hypothesis that SE-1 binds to VirF, we performed thermal shift assays using the dye Sypro Orange. In this assay, there is an increase in fluorescence intensity upon binding of Sypro Orange to the exposed hydrophobic residues of a thermally unfolding protein. The unfolding transition allows calculation of the protein's melting temperature (Tₘ) and any melting temperature changes (ΔTₘ) due to the increased protein stability imparted by ligand binding.

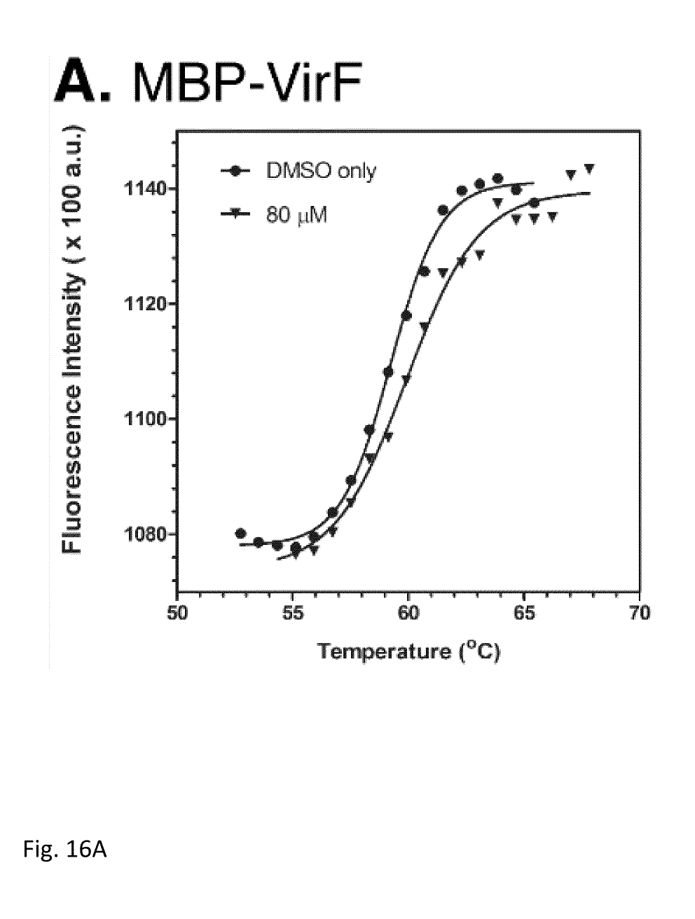

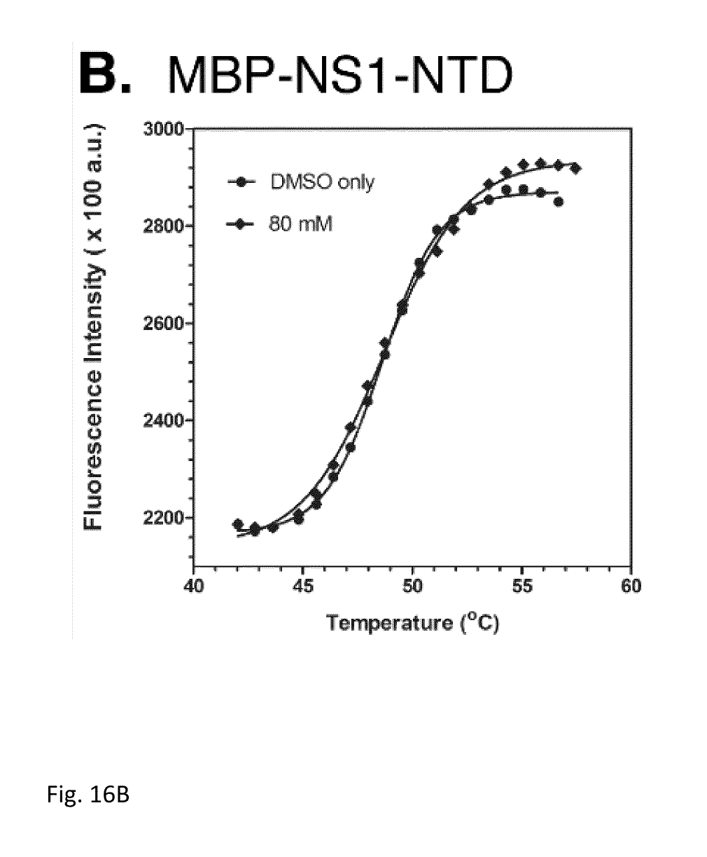

Using the thermal shift assay, we found that addition of 80 μM SE-1 increased the Tₘ of VirF (1 μM) by 0.61° C. (±0.09) (FIG. 16A). While this appears to be a relatively small change in melting temperature, the following support the conclusion that this likely indicates binding of SE-1 to VirF. A cutoff of 0.5° C. or greater melting temperature change can be used to indicate ligand binding to the target protein after testing more than 100 different proteins. Further, the ΔTₘ for VirF with 80 μM SE-1 is much greater than that found with 40 μM SE-1 (0.15° C.±0.04, data not shown), suggesting that VirF is likely not saturated with SE-1 at 80 μM, and that a higher ΔTₘ would likely be found if we could test higher concentrations of SE-1. Finally, there are multiple reports of ΔTₘ values similar to ours that have been validated as true binding to a protein.

Since our VirF protein is a fusion with MBP, we tested an unrelated MBP fusion protein (MBP-NS1-NTD) to determine whether SE-1 bound to MBP. NS1 is a poxvirus protein that is not a member of the AraC family. MBP-NS1-NTD showed only a 0.15° C. (±0.06) change in melting temperature, and the melting curve with SE-1 was not well separated from the DMSO only control (FIG. 16B), suggesting that SE-1 did not bind to MBP. We conclude that SE-1 binds directly to VirF and hypothesize that this binding is responsible for the inhibition of DNA binding and thus, transcription activation, by VirF.

SE-1 Inhibited VirF Activation of a virB-lacZ Fusion in Shigella.

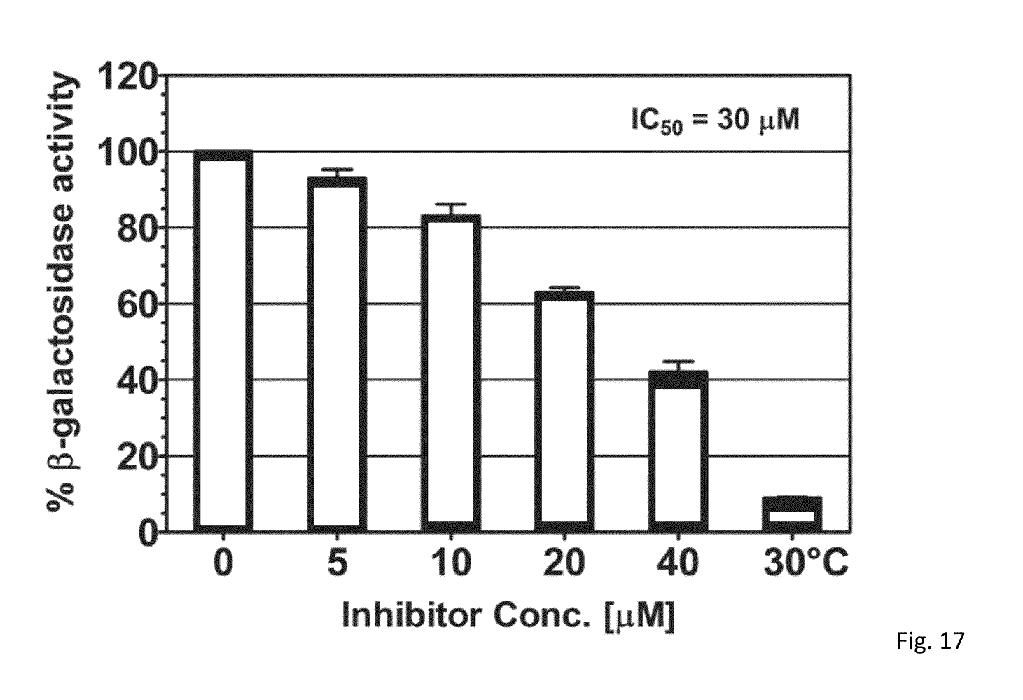

Our cell-based assays in E. coli indicated that SE-1 was able to effectively inhibit activation by VirF at the virB promoter region. To test whether a similar inhibition occurred in Shigella, we assayed the effect of SE-1 on VirF activity in a S. flexneri strain carrying a virB-lacZ fusion. β-galactosidase assays were performed on bacterial cultures grown at 37° C. in the absence or presence of varying concentrations of inhibitor. Our results show a dose-dependent inhibition of virB-lacZ expression, with a maximum inhibition of greater than two fold at 40 μM SE-1 (FIG. 17). This inhibition was most likely due to inhibition of VirF activity. We calculated an IC₅₀ value of approximately 30 μM, which is somewhat higher than the 8 μM IC₅₀ achieved in E. coli. S. flexneri samples grown in 80 μM SE-1 were not analyzed as they exhibited growth defects. Cultures of Shigella grown at 30° C. in the absence of SE-1 were used to identify the basal level of virB-lacZ expression. These assays, similar to the cell-based assays in E. coli, indicate that SE-1 inhibits transcription activation by VirF at the virB-lacZ promoter region, and further provide evidence that SE-1 is effective in Shigella.

SE-1 Reduced VirF-Regulated Virulence Gene Expression in Shigella.

Our next goal was to test the impact of SE-1 on expression of other VirF-regulated genes in Shigella. Previous reports have shown that icsA and virB genes are direct targets of VirF activation at 37° C. VirB, in turn, activates the expression of many operons that play a crucial role in Shigella pathogenesis. Thus, any compound that inhibits transcription activation by VirF should also affect the expression levels of genes that either directly or indirectly require VirF for their expression. To test this hypothesis, we used qRT-PCR to quantify mRNA levels of two direct VirF target genes, icsA and virB, and two indirect VirF target genes, icsB and ipaB (both directly activated by VirB). S. flexneri samples were grown to an OD₆₀₀ of ˜1.0 in the absence or presence of two different concentrations of SE-1 (20 μM, 40 μM) and the mRNA levels from each of these genes were quantified. S. flexneri samples grown at 30° C. in the absence of SE-1 were included to illustrate the basal expression levels of expression of these genes. Results were normalized to the gapA (encodes glyceraldehyde-3-phosphate dehydrogenase) and rrsA (encodes 16S rRNA) genes, which are two constitutively expressed genes that are commonly used as qRT-PCR controls in Shigella.

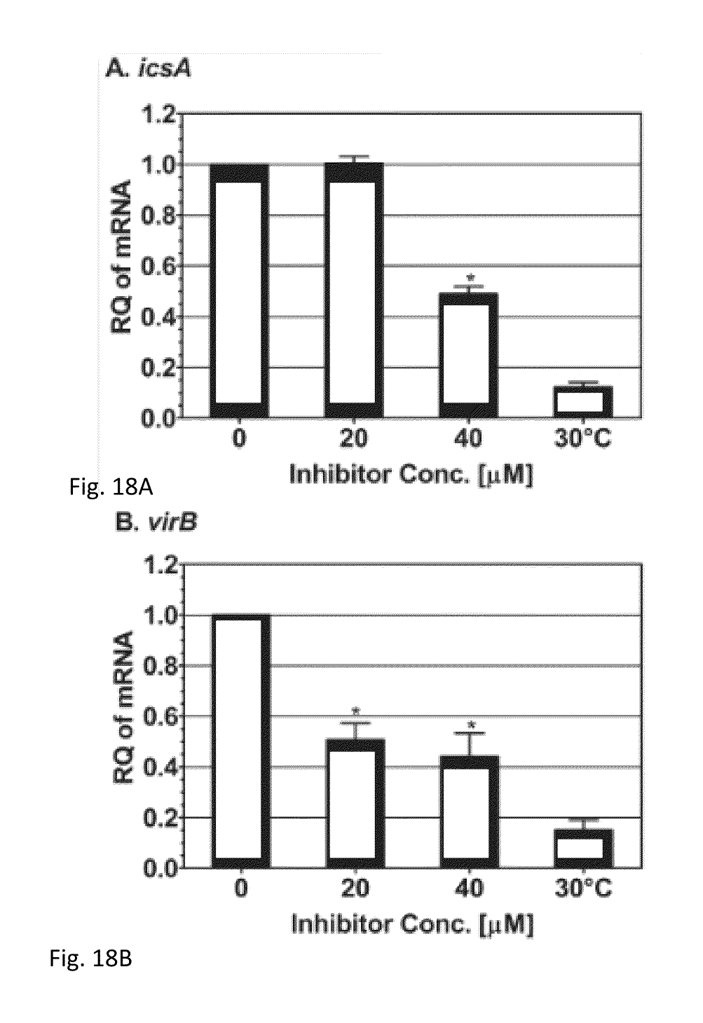

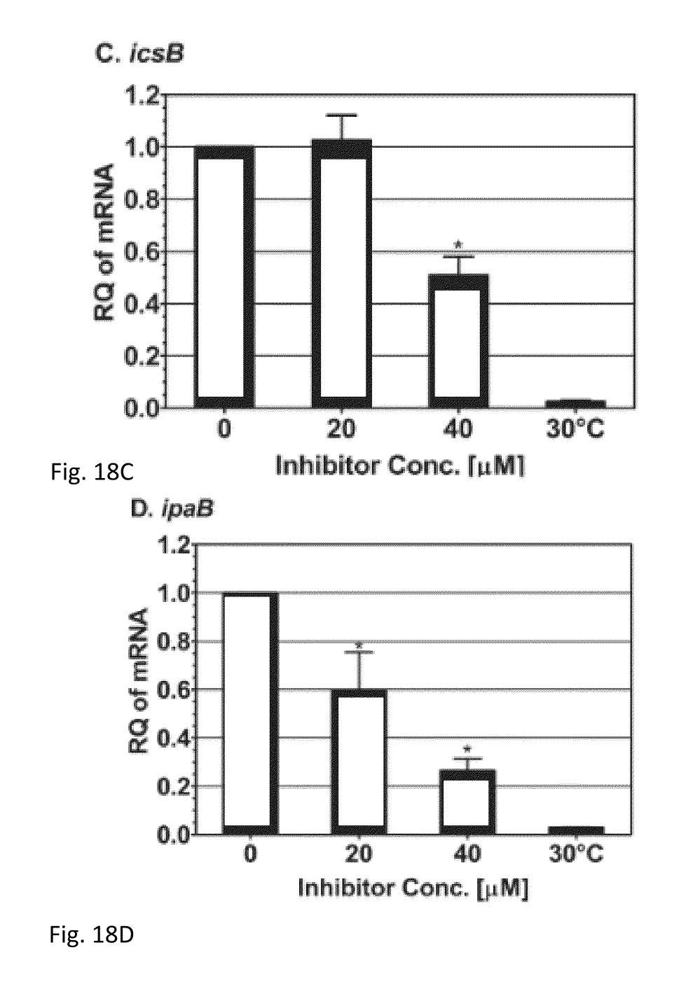

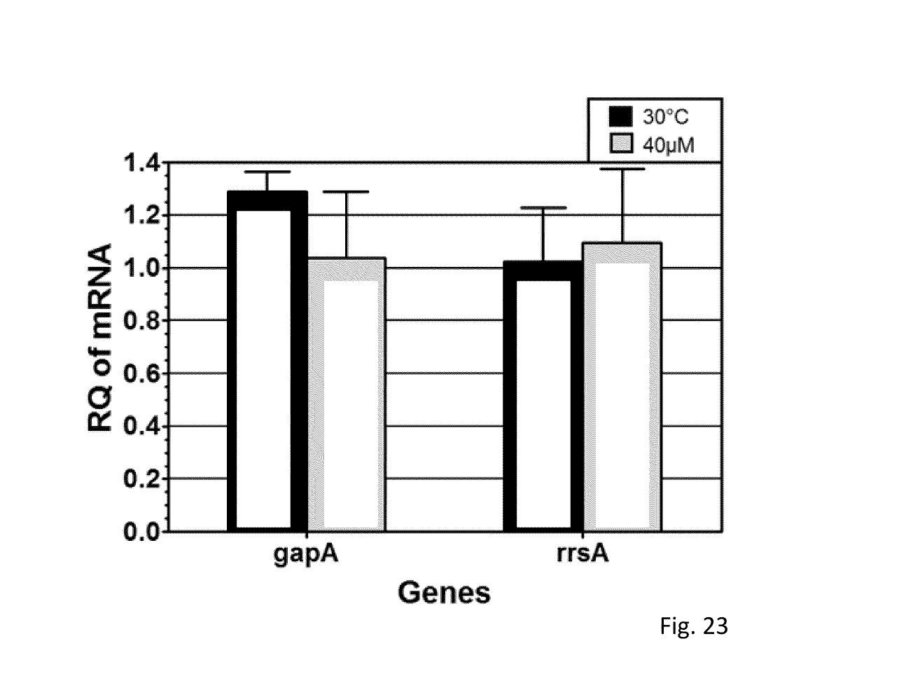

Our qRT-PCR results showed that the icsA expression level remained essentially unchanged at 20 μM inhibitor, but was significantly reduced (P<0.05) at 40 μM inhibitor (FIG. 18A). Expression of virB, another direct target of VirF exhibited a significant decrease in expression with increasing concentrations of inhibitor, and a maximal inhibition of more than two fold at 40 μM SE-1 (P<0.05) (FIG. 18B). Expression of icsB, an indirect target of VirF, showed a significant two-fold reduction at 40 μM SE-1 (P<0.05) (FIG. 18C). The greatest inhibition was detected for expression of ipaB, another indirect target of VirF, which was reduced by up to 4-fold at 40 μM inhibitor (P<0.05) (FIG. 18D). As a control, we normalized each of the internal control genes relative to the other internal control gene. We found that there was no decrease in the expression of either control gene with 40 μM SE-1 (FIG. 23), arguing that SE-1 did not result in a global decrease in gene expression. Together, our data demonstrate that SE-1 is capable of reducing the expression of at least four VirF-regulated virulence-associated genes in Shigella, presumably through its inhibition of transcription activation by VirF.

SE-1 Inhibited Host Cell Invasion by Shigella.

Our qRT-PCR experiments with SE-1 showed a decrease in expression of virulence genes in Shigella. In order to test the impact of this decrease in virulence gene expression on the ability of Shigella to invade host cells, invasion assays were performed to evaluate the effect of SE-1 on Shigella invasion of the mouse fibroblast cell line L-929.

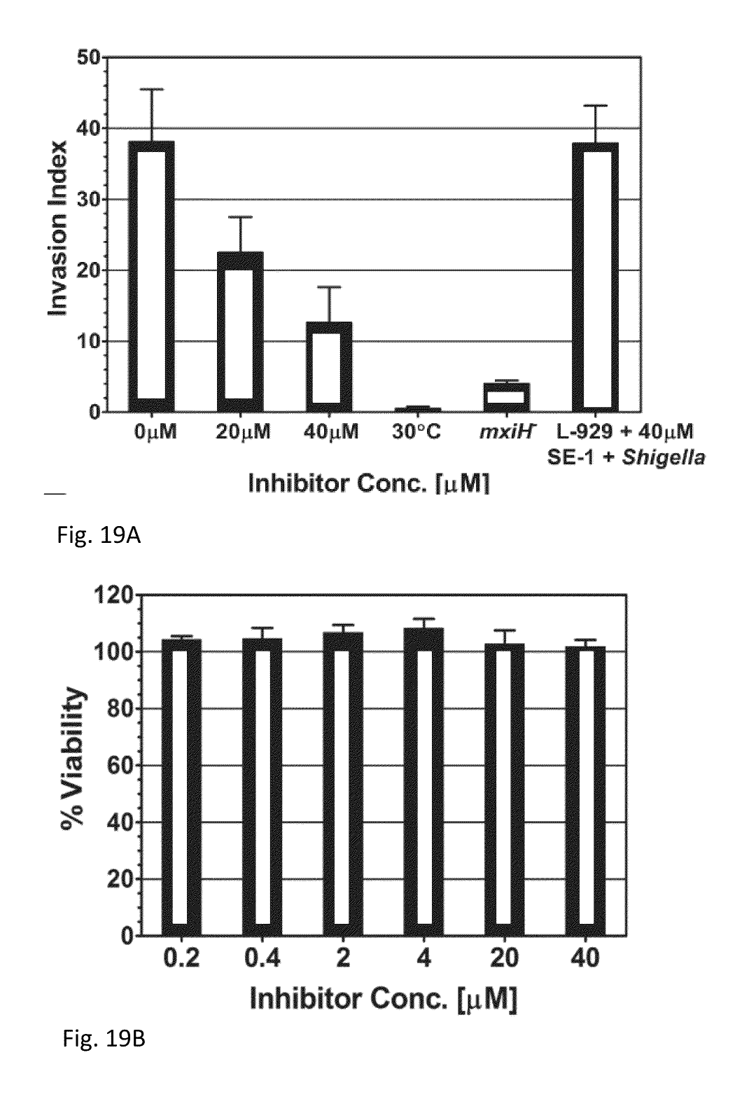

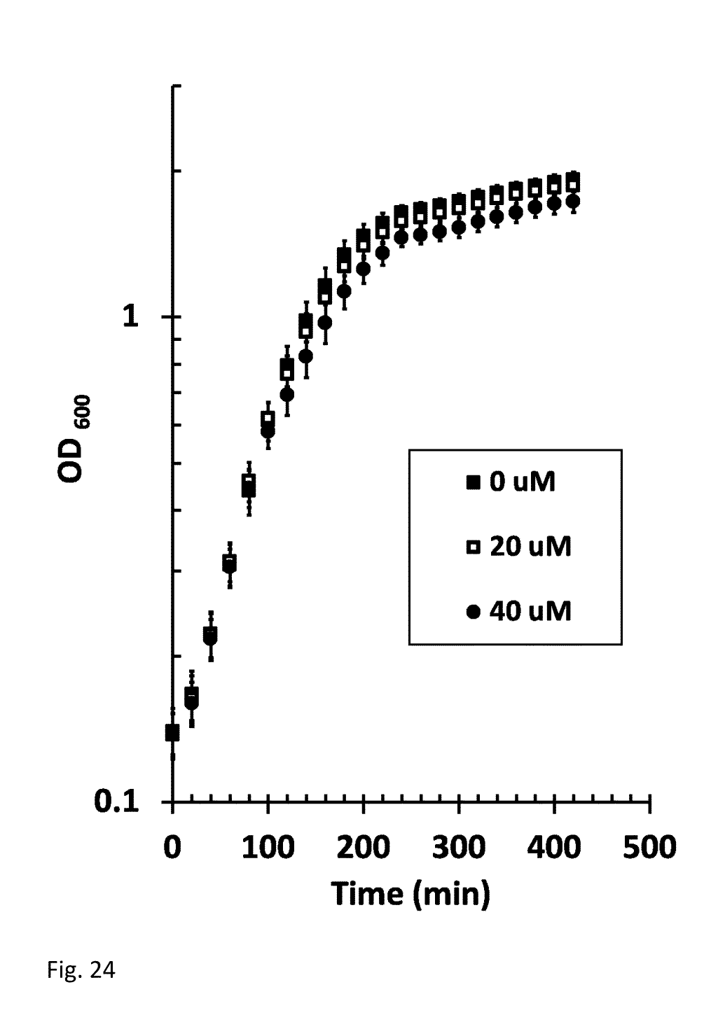

L-929 cells have been used to study invasion of Shigella into host cells. The S. flexneri strain used in the assays was an ipgD strain, which has hemolysis and invasion properties that are similar to wild type, but is safer to work with due to its reduced ability to cause human infection. The cells were grown at 30° C. overnight and then diluted and grown at 37° C. (to induce expression of VirF-regulated genes) in the presence or absence of various concentrations of SE-1. In addition, S. flexneri strain SB116 (mxiH⁻) was used as a negative control for invasion since it does not form a T3SS needle and thereby is unable to invade host cells. S. flexneri grown at 30° C. was used as a second negative control for invasion, as these bacteria have reduced expression of VirF and VirB and thereby are defective for invasion of host cells. Our results showed a dose-dependent decrease in invasion of Shigella into L-929 cells upon addition of SE-1 (FIG. 19A). SE-1 resulted in invasion decreases of 1.7-fold at 20 μM and 3-fold at 40 μM relative to no inhibitor. As expected, both of the negative controls (wild-type S. flexneri grown at 30° C. and the mxiH⁻ strain) showed substantially decreased invasion relative to the wild-type strain grown at 37° C. Similar to our growth assays in E. coli, we found that 0.3% DMSO did not detectably slow the growth of Shigella (data not shown), and SE-1 resulted in no detectable decrease in the Shigella growth rate at concentrations up to 40 μM (FIG. 24).

Overall, our results suggest the hypothesis that SE-1 inhibits Shigella invasion by decreasing the expression of the genes in the VirF regulon, including the genes that encode the T3SS. Thus, Shigella grown under inducing conditions (37° C.) without SE-1 would not be inhibited by addition of SE-1 unless growth with SE-1 was continued for a period of time that was sufficient for loss of preformed VirF-regulated gene products, such as the T3SS. To test this hypothesis, Shigella were grown at 37° C. in the absence of inhibitor, and then Shigella and SE-1 were added at the same time to L-929 cells. This control also tested whether SE-1 had any impact on the L-929 cells that affected invasion by Shigella. Our result suggest that the inhibition of invasion by SE-1 was likely not due to post-transcriptional effects on Shigella or effects on the eukaryotic cells, as this control sample invaded to the same extent as Shigella grown in the absence of inhibitor (FIG. 19A). Overall, our results support the hypothesis that SE-1 decreases the expression of VirF-regulated virulence genes, and that this decrease in turn resulted in a reduction in the ability of Shigella to invade host cells.

The effect of SE-1 on the viability of L-929 cells was assayed using an AlamarBlue® cell viability assay (Invitrogen, Grand Island, N.Y.). The AlamarBlue® assay is a fluorescence-based assay of the viability of host cells based on their metabolic activity. During our invasion assays, the L-929 cells were only exposed to SE-1 concentrations of 0.2 and 0.4 μM, due to dilution of the SE-1-containing Shigella culture into the invasion assays. Our AlamarBlue® assays indicated that these concentrations of SE-1 did not detectably decrease the viability of L-929 cells (FIG. 19B). In fact, a concentration 100-fold higher than this (40 μM) had no effect on the host cell viability. These results support the hypothesis that the effect of SE-1 on invasion by Shigella was not an indirect effect on L-929 cell viability. Further, the results provide evidence that SE-1 did not have detectable toxicity toward the L-929 cells at concentrations up to 40 μM.

Accordingly, the compounds of the invention can inhibit various bacterial pathogens that require AraC family transcriptional regulators to cause disease. VirF, one such AraC family regulator, activates the expression of a cascade of virulence genes required for successful infection by Shigella, and it can be inhibited by the compounds of the invention. Thus, the compounds of the invention can be inhibitors that block transcription activation by VirF (preferably without detrimental effects on growth of the bacteria) have potential to be developed into novel antimicrobial agents. The non-essential nature of VirF may reduce the probability that Shigella will develop resistance to VirF inhibitors, and thereby the compound of the invention can be used in methods for inhibiting disease without inducing or causing the microbes of the disease to have resistance thereto. We recently identified an inhibitor, SE-1, which inhibited transcription activation by the AraC family proteins RhaS and RhaR by blocking their ability to bind to DNA. In the present study, we tested SE-1 for inhibition of VirF and VirF-dependent Shigella virulence.

Using a cell-based reporter gene assay to test the ability of SE-1 to inhibit transcription activation by the VirF protein, we found that SE-1 decreased virB-lacZ reporter fusion expression in an E. coli strain with plasmid-expressed VirF. The inhibition was dose-dependent and had an IC₅₀ value of approximately 8 μM, suggesting that SE-1 inhibited transcription activation by VirF. An initial indication that this inhibition was selective for VirF activity was the finding that SE-1 was substantially less inhibitory toward lacZ expression from a control fusion (hts-lacZ) than VirF-dependent lacZ expression. The strains carrying the VirF-dependent and control lacZ fusions showed no detectable growth defects in the presence of SE-1, indicating that SE-1 did not have general toxicity toward these E. coli strains at the concentrations tested.

We showed that the SE-1 inhibition of the AraC family activators RhaS and RhaR involved blocking binding to DNA. Thus, we tested SE-1 for inhibition of DNA binding of purified VirF protein to DNA carrying a VirF binding site, and found that SE-1 was able to fully inhibit DNA binding by VirF. We showed that SE-1 did not inhibit DNA binding by two proteins that are not related to the AraC family, Lad and CRP, suggesting that SE-1 is selective for AraC family proteins. Our thermal shift assay results suggest that SE-1 can bind directly to the VirF protein. Overall, our results support the hypothesis that the mechanism of action of SE-1 inhibition involves selectively blocking DNA binding by VirF by directly binding to the protein.

We next tested inhibition by SE-1 in Shigella, initially of endogenously expressed VirF in a Shigella strain carrying a virB-lacZ reporter construct. Similar to our findings in E. coli, we observed dose-dependent inhibition of VirF activation of virB-lacZ in Shigella. In addition, Shigella grown in the presence of SE-1 showed a decrease in transcription (assayed by qRT-PCR) of genes that are directly activated by VirF (virB, icsA) as well as genes that are directly activated by VirB (indirect VirF targets, icsB, ipaB). In these experiments, we observed up to two- to four-fold decreases in expression of these VirF-dependent genes, with values consistent with the virB-lacZ assays in Shigella. However, the same concentration of SE-1 resulted in lower levels of inhibition in Shigella than in E. coli, suggesting differences in uptake, efflux and/or stability of SE-1 in these two bacteria.

Finally, we investigated the effect of SE-1 on the ability of Shigella to invade mouse fibroblast (L-929) cells in tissue culture. A dose-dependent decrease in invasion was observed with a maximum inhibition of 70% at 40 μM inhibitor. This inhibition required pre-incubation of Shigella with SE-1, consistent with the hypothesis that SE-1 did not destabilize preassembled T3SS or other Shigella virulence components, inhibit post-transcriptional processes in Shigella, or cause cytotoxicity to the L-929 cells at the concentrations tested. The latter is further supported by the finding that SE-1 did not result in a detectable decrease in metabolism of L-929 cells in AlamarBlue® assays at these same concentrations.

Overall, our results suggest a model in which SE-1 inhibits VirF-dependent transcription activation of Shigella virulence genes, including at least icsA, virB, icsB and ipaB. The decrease in invasion we detected is consistent with and can be attributed to the effect of decreased expression of these virulence genes in the VirF regulon. The two- to four-fold decreases in expression of these genes appear to be sufficient to decrease invasion of Shigella by 70%, compared to the uninhibited invasion level. The ipaB gene exhibited the largest decrease in expression, four-fold, suggesting that reduction in IpaB expression may have limited invasion in the presence of SE-1. This is a reasonable hypothesis given the central role IpaB plays in Shigella invasion. IpaB is a component of the translocon pores in the host cell membrane that enable translocation of Shigella effectors into host cells, ultimately modulating host cell cytoskeletal dynamics and leading to bacterial invasion. In addition, only a subset of the VirF-regulated virulence genes that are required for invasion were assayed, thus, decreased expression of other genes likely also contributed to the invasion defects.

We have now shown that SE-1 selectively inhibits DNA binding by three different AraC family proteins. The VirF protein shares only about 15% amino acid sequence identity and 40% similarity with RhaS and RhaR (the pairwise RhaS-VirF and RhaR-VirF comparisons are nearly the same), see incorporated references. Given our finding that SE-1 blocks transcription activation by the RhaS DBD to at least the same extent as the full-length RhaS protein, we hypothesize that the VirF DNA binding domain is the likely site of action of SE-1. The sequence comparisons in this domain are somewhat higher than the full-length proteins, but are still relatively low, with the VirF DBD sharing about 22% identity and 52% similarity with the RhaS and RhaR DBDs. This finding supports the hypothesis that SE-1 may bind to a relatively conserved region of AraC family DNA binding domains. In addition to the inhibitory effects of SE-1 on VirF from the S. flexneri strains used in our work, SE-1 may also be effective against other pathogenic Shigella species (S. dysenteriae, S. sonnei and S. boydii), since VirF is the master virulence regulator of virulence in all these species and shares ˜100% identity.

Also, it is likely that analogs of SE-1 can be used as inhibitors of transcription activation by VirF using cell-based reporter gene assays, DNA binding assays and invasion assays. The compounds are likely not non-cytotoxic toward the eukaryotic cells tested (at concentrations up to 40 μM) and non-bactericidal toward E. coli and Shigella (at concentrations up to 40 μM).

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety: Koppolu, V., I. Osaka, J. M. Skredenske, B. Kettle, P. S. Hefty, J. Li and S. M. Egan. 2013. Small Molecule Inhibitor of Shigella flexneri Master Virulence Regulator, VirF. Infection and Immunity. Published ahead of print 3 Sep. 2013, doi:10.1128/IAI.00919-13; and Skredenske, J. M., V. Koppolu, A. Kolin, J. Deng, B. Kettle, B. Taylor, and S. M. Egan. 2013. Identification of a Small Molecule Inhibitor of Bacterial AraC Family Activators. Journal of Biomolecular Screening. 18(5):588-598.

TABLE S1
Analysis of example real-time PCR gene expression data.

| | | | | | Average | RQ |
|---|---|---|---|---|---|---|
| Gene | SE-1 (μM) | Ct | ΔΔCt¹ | ΔΔCt² | ΔΔCt | (2^{-ΔΔCt}) |
| gapA | 0 | 26.70 | | 0 | 0 | 1 |
| gapA | 20 | 27.33 | | 1 | 1 | 0.5 |
| gapA | 40 | 26.76 | | −0.69 | −0.69 | 0.62 |
| rrsA | 0 | 18.91 | 0 | | 0 | 1 |
| rrsA | 20 | 18.55 | −1.0 | | −1.0 | 2 |
| rrsA | 40 | 18.29 | 0.69 | | 0.69 | 1.62 |
| virB | 0 | 22.00 | 0 | 0 | 0 | 1 |
| virB | 20 | 23.26 | 0.63 | 1.63 | 1.13 | 0.46 |
| virB | 40 | 23.18 | 1.12 | 1.81 | 1.47 | 0.36 |
| icsA | 0 | 27.70 | 0 | 0 | 0 | 1 |
| icsA | 20 | 27.88 | −0.46 | 0.54 | 0.04 | 0.97 |
| icsA | 40 | 28.59 | 0.82 | 1.52 | 1.17 | 0.44 |
| icsB | 0 | 26.17 | 0 | 0 | 0 | 1 |
| icsB | 20 | 26.25 | −0.56 | 0.44 | −0.06 | 1.04 |
| icsB | 40 | 27.04 | 0.80 | 1.49 | 1.14 | 0.45 |
| ipaB | 0 | 24.32 | 0 | 0 | 0 | 1 |
| ipaB | 20 | 25.57 | 0.62 | 1.61 | 1.11 | 0.46 |
| ipaB | 40 | 26.23 | 1.85 | 2.54 | 2.20 | 0.22 |

¹ΔΔCt values after normalizing data to gapA;
²ΔΔCt values after normalizing data to rrsA
ΔΔCt = (Ct, Target - Ct, Internal Control) SE-1 - (Ct, Target - Ct, Internal Control) no SE-1

### Compounds of the Invention

| | | % Inhibition at 82.5 μM | | |
|---|---|---|---|---|
| Compound # | Structure | RhaS | Rns | VirF |
| Compound 1 | 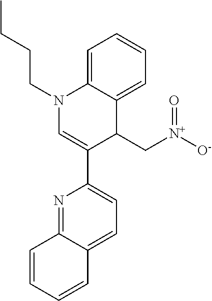 [SMILES: CCCCN1C=C(c2ccc3ccccc3n2)C(C[N+](=O)[O-])c2ccccc21] | 90 | 90 | 100 |
| Compound 2 | 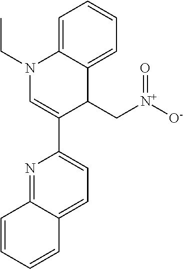 [SMILES: CCN1C=C(c2ccc3ccccc3n2)C(C[N+](=O)[O-])c2ccccc21] | 90 | 60 | 100 |
| JS01 | 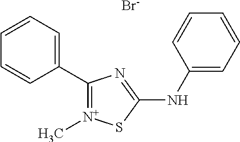 [SMILES: [Br-] ; C[n+]1sc(Nc2ccccc2)nc1-c1ccccc1] | 100 | 100 | 100 |
| A2 | 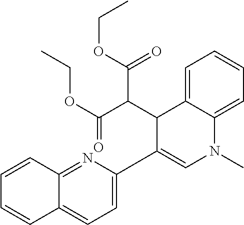 [SMILES: CCOC(=O)C(C(=O)OCC)C1C(c2ccc3ccccc3n2)=CN(C)c2ccccc21] | 80 | 80 | 100 |
| E1 | 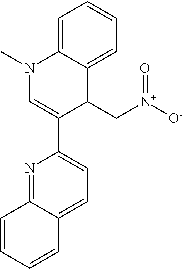 [SMILES: CN1C=C(c2ccc3ccccc3n2)C(C[N+](=O)[O-])c2ccccc21] | 90 | 80 | 100 |
| A24 | 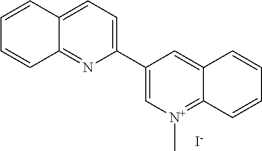 [SMILES: C[n+]1cc(-c2ccc3ccccc3n2)cc2ccccc21 ; [I-]] | 94 | 90 | 100 |
| A25 | 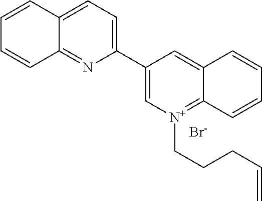 [SMILES: C=CCCC[n+]1cc(-c2ccc3ccccc3n2)cc2ccccc21 ; [Br-]] | 80 | 100 | 100 |
| I10 | 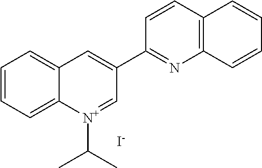 [SMILES: CC(C)[n+]1cc(-c2ccc3ccccc3n2)cc2ccccc21 ; [I-]] | 80 | 90 | 100 |
| I11 | 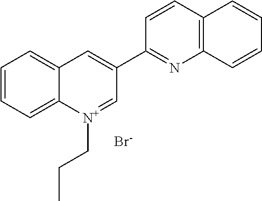 [SMILES: CCC[n+]1cc(-c2ccc3ccccc3n2)cc2ccccc21 ; [Br-]] | 80 | 90 | 100 |
| I12 | 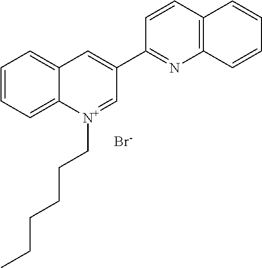 [SMILES: CCCCCC[n+]1cc(-c2ccc3ccccc3n2)cc2ccccc21 ; [Br-]] | 90 | 100 | 100 |
| I13 | 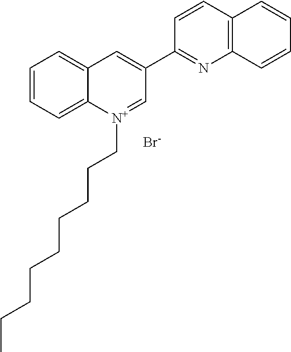 [SMILES: CCCCCCCCC[n+]1cc(-c2ccc3ccccc3n2)cc2ccccc21 ; [Br-]] | 100 | 100 | 100 |
| I14 | 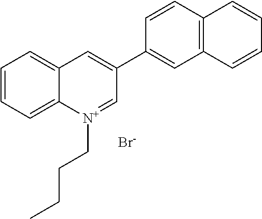 [SMILES: CCCC[n+]1cc(-c2ccc3ccccc3c2)cc2ccccc21 ; [Br-]] | 90 | 100 | 100 |
| C4 | 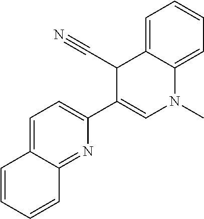 [SMILES: CN1C=C(c2ccc3ccccc3n2)C(C#N)c2ccccc21] | 70 | 80 | 90 |
| A07 | 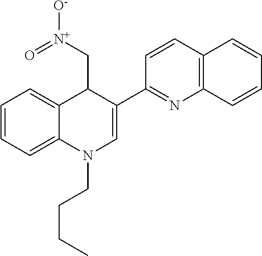 [SMILES: CCCCN1C=C(c2ccc3ccccc3n2)C(C[N+](=O)[O-])c2ccccc21] | 90 | 90 | 90 |
| I02 | 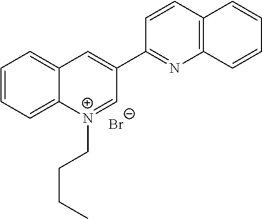 [SMILES: CCCC[n+]1cc(-c2ccc3ccccc3n2)cc2ccccc21 ; [Br-]] | 90 | 90 | 90 |
| D3 | 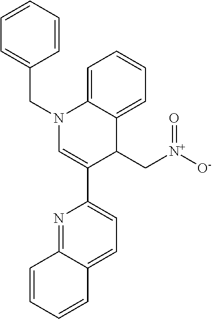 [SMILES: O=[N+]([O-])CC1C(c2ccc3ccccc3n2)=CN(Cc2ccccc2)c2ccccc21] | 60 | 60 | 70 |
| A21 | 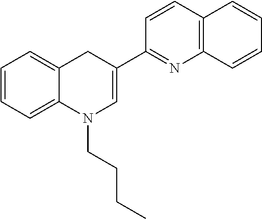 [SMILES: CCCCN1C=C(c2ccc3ccccc3n2)Cc2ccccc21] | 60 | 80 | 70 |
| I03 | 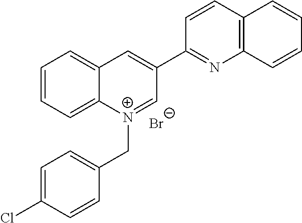 [SMILES: Clc1ccc(C[n+]2cc(-c3ccc4ccccc4n3)cc3ccccc32)cc1 ; [Br-]] | 60 | 50 | 60 |
| JS03 | 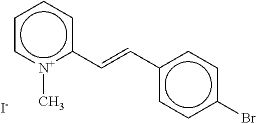 | 20 | 10 | 50 |
| I01 | 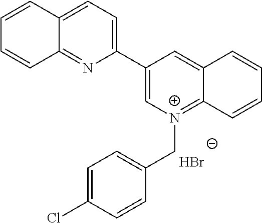 [SMILES: Clc1ccc(C[n+]2cc(-c3ccc4ccccc4n3)cc3ccccc32)cc1 ; [Br-]] | 50 | 50 | 50 |
| D2 | 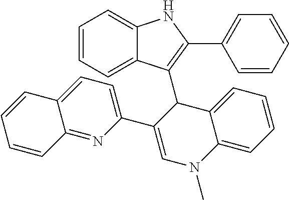 [SMILES: CN1C=C(c2ccc3ccccc3n2)C(c2c(-c3ccccc3)[nH]c3ccccc23)c2ccccc21] | 10 | 10 | 40 |
| A08 | 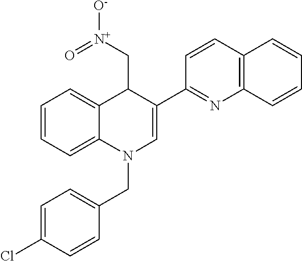 [SMILES: O=[N+]([O-])CC1C(c2ccc3ccccc3n2)=CN(Cc2ccc(Cl)cc2)c2ccccc21] | 40 | 30 | 40 |
| A23 | 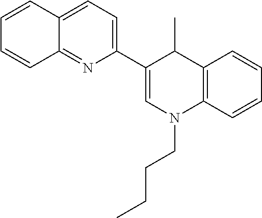 [SMILES: CCCCN1C=C(c2ccc3ccccc3n2)C(C)c2ccccc21] | 0 | 10 | 30 |
| A1 | 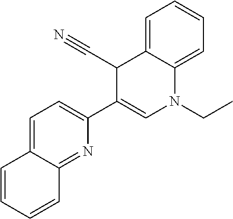 [SMILES: CCN1C=C(c2ccc3ccccc3n2)C(C#N)c2ccccc21] | 30 | 30 | 20 |
| A01 | 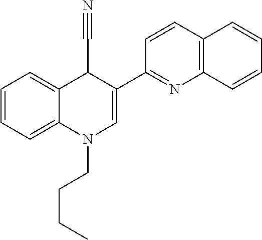 [SMILES: CCCCN1C=C(c2ccc3ccccc3n2)C(C#N)c2ccccc21] | 30 | 20 | 20 |
| A3 | 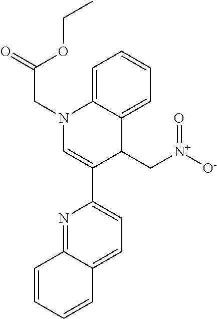 [SMILES: CCOC(=O)CN1C=C(c2ccc3ccccc3n2)C(C[N+](=O)[O-])c2ccccc21] | 0 | 30 | 10 |
| A09 | 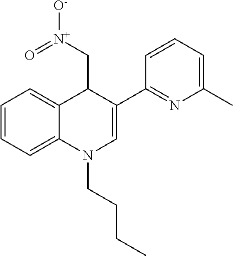 [SMILES: CCCCN1C=C(c2cccc(C)n2)C(C[N+](=O)[O-])c2ccccc21] | 50 | 0 | 10 |
| A10 | 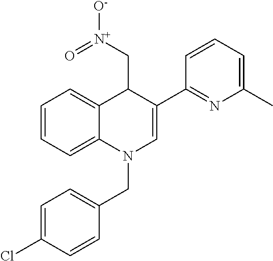 [SMILES: Cc1cccc(C2=CN(Cc3ccc(Cl)cc3)c3ccccc3C2C[N+](=O)[O-])n1] | 30 | 0 | 10 |
| A03 | 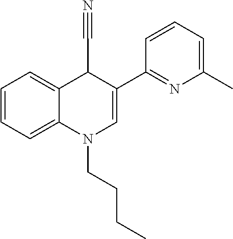 [SMILES: CCCCN1C=C(c2cccc(C)n2)C(C#N)c2ccccc21] | 40 | 10 | 0 |

In one embodiment, the invention can omit or exclude the following compounds, and the compounds of the invention can omit the R groups of these compounds in the corresponding formulae of Formula 1, Formula 2, or Formula 3.

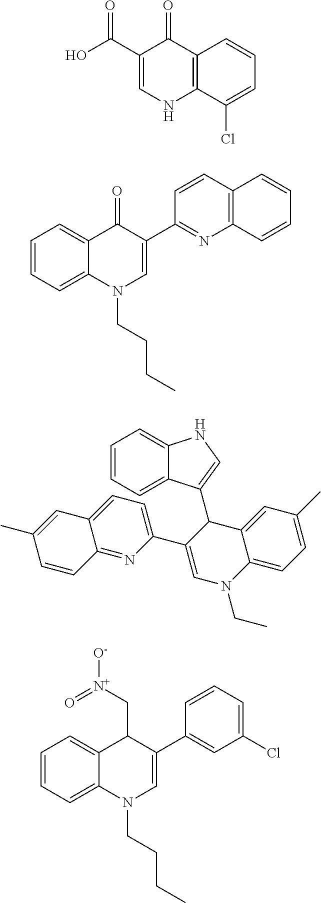

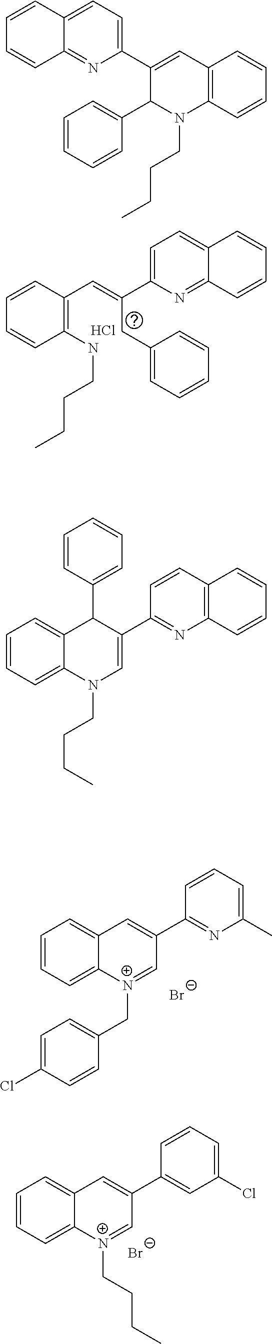

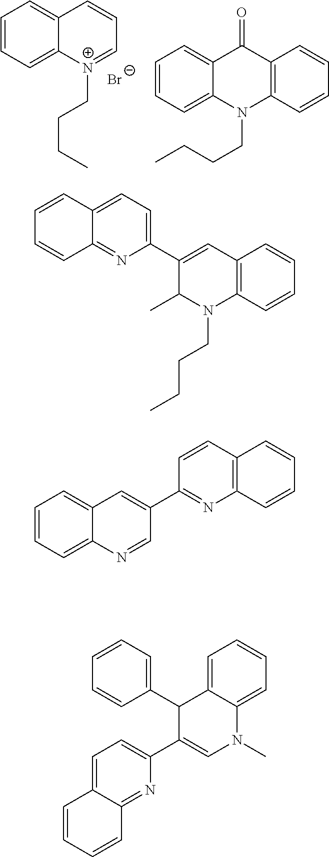

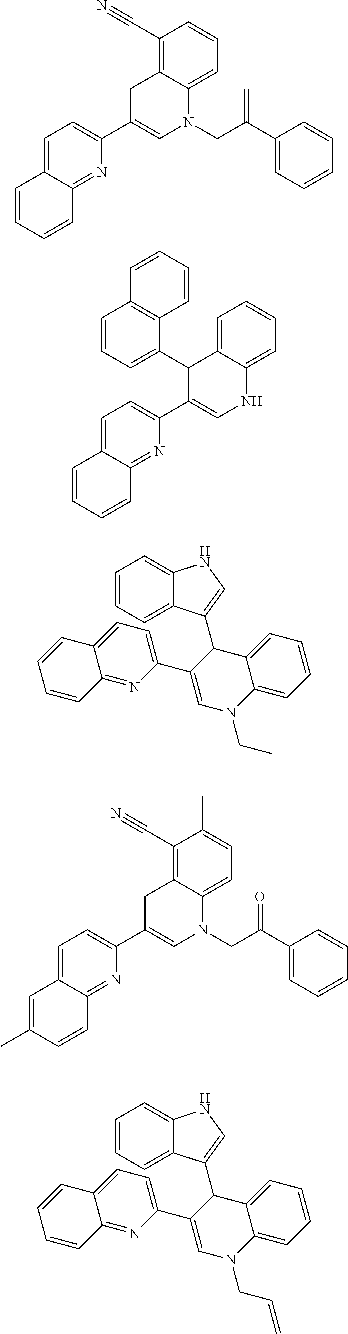 [SMILES: C=C(CN1C=C(c2ccc3ccccc3n2)Cc2c(C#N)cccc21)c1ccccc1 ; C1=C(c2ccc3ccccc3n2)C(c2cccc3ccccc23)c2ccccc2N1 ; CCN1C=C(c2ccc3ccccc3n2)C(c2c[nH]c3ccccc23)c2ccccc21 ; Cc1ccc2nc(C3=CN(CC(=O)c4ccccc4)c4ccc(C)c(C#N)c4C3)ccc2c1 ; C=CCN1C=C(c2ccc3ccccc3n2)C(c2c[nH]c3ccccc23)c2ccccc21]

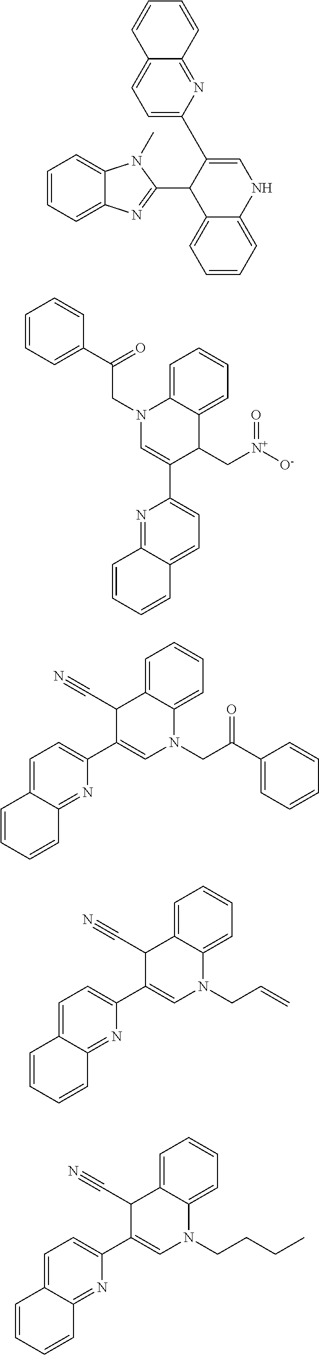

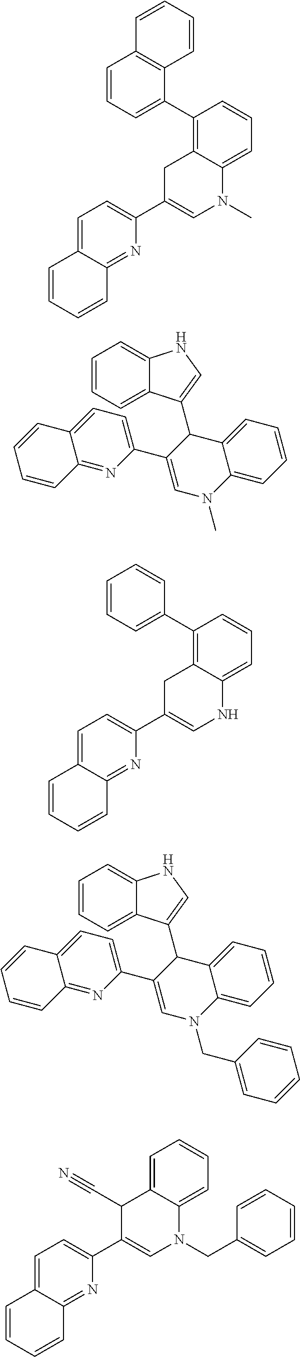

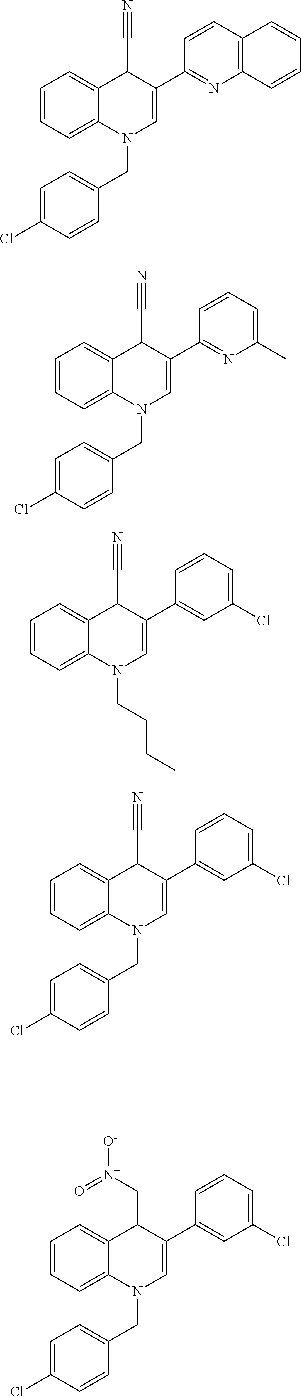

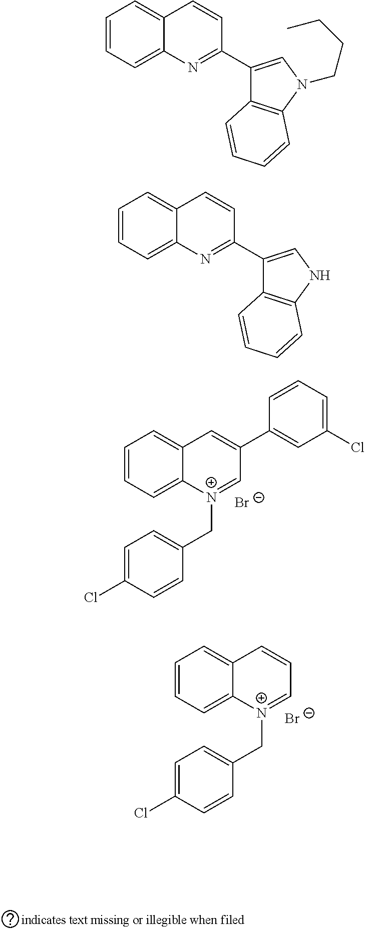

1. A method of inhibiting a microbial infection, the method comprising:
providing a compound represented by Formula 1 or prodrugs or pharmaceutically acceptable salts thereof; and
administering the compound to a subject in a therapeutically effective amount to inhibit the microbial infection, 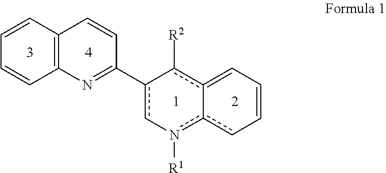
wherein:
R¹ includes hydrogen, halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, derivatives thereof, substituted or unsubstituted, or combinations thereof;
R² includes hydrogen, halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, derivatives thereof, substituted or unsubstituted, or combinations thereof;
wherein the nitrogen of ring 1 includes 3 or 4 bonds, when having 4 bonds a counter anion is present; and
wherein ring 2 is aromatic.

2. The method of claim 1, wherein the therapeutically effective amount is sufficient to inhibit a biological activity of a transcriptional activator of the microbe.

3. The method of claim 2, wherein the inhibited transcriptional activator is an AraC bacterial transcriptional activator.

4. The method of claim 3, wherein the AraC bacterial transcriptional activator is RhaS.

5. The method of claim 3, wherein the AraC bacterial transcriptional activator is RhaR.

6. The method of claim 3, wherein the AraC bacterial transcriptional activator is Rns.

7. The method of claim 3, wherein the AraC bacterial transcriptional activator is VirF.

8. The method of claim 3, wherein the microbe is selected from Vibrio, Pseudomonas, Enterotoxigenic E. coli, and Shigella.

9. The method of claim 1, comprising inhibiting diarrhea associated with the microbial infection.

10. The method of claim 1, wherein the administration is prior to infection with the microbe.

11. The method of claim 1, wherein the administration is after infection with the microbe.

12. The method of claim 1, wherein the compound has specific inhibition of AraC bacterial transcriptional activators over other transcriptional activators.

13. The method of claim 1, wherein the therapeutically effective amount is sufficient to inhibit a virulence factor of the microbe.

14. The method of claim 1, wherein the therapeutically effective amount is sufficient to inhibit microbe entry into a cell, wherein the microbe is Shigella.

15. The method of claim 1, wherein the therapeutically effective amount is not toxic to the microbe.

16. The method of claim 2, wherein the compound sterically inhibits the transcriptional factor from binding with DNA.

17. The method of claim 1, wherein the therapeutically effective amount is sufficient to reduce expression of VirF-dependent virulence genes.

18. The method of claim 17, wherein the VirF-dependent virulence genes are selected from icsA, virB, icsB and ipaB.

19. A method of inhibiting a transcriptional factor from transcribing DNA, the method comprising:
providing a compound represented by Formula 1 and prodrugs and pharmaceutically acceptable salts thereof; and
administering the compound to a transcriptional factor in a therapeutically effective amount to inhibit the transcriptional factor from binding DNA and thereby preventing transcription of the genes regulated by the transcription factor, 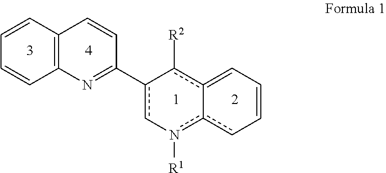
wherein:
R¹ includes hydrogen, halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, derivatives thereof, substituted or unsubstituted, or combinations thereof;
R² includes hydrogen, halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, derivatives thereof, substituted or unsubstituted, or combinations thereof;
wherein the nitrogen of ring 1 includes 3 or 4 bonds, when having 4 bonds a counter anion is present; and
wherein ring 2 is aromatic.

20. A method of inhibiting a microbe from entering a cell of a subject, the method comprising:
providing a compound represented by Formula 1 an prodrugs and pharmaceutically acceptable salts thereof; and
administering the compound to the subject having the cell in a therapeutically effective amount to inhibit the microbe from entering the cell, 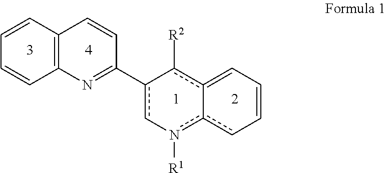
wherein:
R¹ includes hydrogen, halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, derivatives thereof, substituted or unsubstituted, or combinations thereof;
R² includes hydrogen, halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, amines, primary amines, secondary amines, tertiary amines, aliphatic amines, carbonyls, carboxyls, amides, esters, amino acids, peptides, polypeptides, derivatives thereof, substituted or unsubstituted, or combinations thereof;
wherein the nitrogen of ring 1 includes 3 or 4 bonds, when having 4 bonds a counter anion is present; and
wherein ring 2 is aromatic.
